SEQUENCE LISTING (6 sequences)

SEQ ID NO: 1 - DNA, 20 bp, artificial sequence
  primer
ccatccgacattgaagttga

SEQ ID NO: 2 - DNA, 20 bp, artificial sequence
  primer
tggagcaacctgctcagata

SEQ ID NO: 3 - DNA, 21 bp, artificial sequence
  primer
tggtatcgtggaaggactcat

SEQ ID NO: 4 - DNA, 21 bp, artificial sequence
  primer
gtgggtgtcgctgttgaagtc

SEQ ID NO: 5 - DNA, 43 bp, artificial sequence
  primer
aaacgacggccagtgaattgtaatacgactcactataggcgct

SEQ ID NO: 6 - DNA, 30 bp, artificial sequence
  primer
aagcagtggtatcaacgcagagtacgcggg

(19) United States
(12) Patent Application Publication
(10) Pub. No.: US 2004/0127445 A1
(43) Pub. Date: Jul. 1, 2004

(54) Beta-2 microglobulin (B2M) and B2M related gene products for the regulation of osteoarthritis pathogenesis and chondrocyte proliferation

(75) Inventors: Choong-Chin Liew, Toronto (CA); K. Wayne Marshall, Toronto (CA); Hongwei Zhang, Toronto (CA)

Correspondence Address: PALMER & DODGE, LLP, KATHLEEN M. WILLIAMS, 111 HUNTINGTON AVENUE, BOSTON, MA, 02199, US

(73) Assignee: Chondrogene Limited

(21) Appl. No.: 10/649,959

(22) Filed: Aug. 26, 2003

Related U.S. Application Data
(60) Provisional application No. 60/406,494, filed on Aug. 28, 2002.

(51) Int. Cl. C12Q 1/68; G01N 33/53; A61K 48/00; A61K 38/17

(52) U.S. Cl. 514/44; 514/12; 530/350; 536/23.1; 435/6; 435/7.1

(57) ABSTRACT

The invention relates to the discovery of the role of beta-2 microglobulin (B2M) in the pathogenesis of osteoarthritis (OA) and the ability of B2M to inhibit chondrocyte proliferation. The invention further relates to the identification of genes regulated by B2M (the “B2M related genes”).

### RELATED APPLICATION

This application claims the benefit of U.S. Provisional Application No. 60/406,494 filed on Aug. 28, 2002. The entire teachings of the above application are incorporated herein by reference.

### FIELD OF THE INVENTION

The invention relates to the discovery of the role of beta-2 microglobulin (B2M) in the pathogenesis of osteoarthritis (OA) and the ability of B2M to inhibit chondrocyte proliferation. Also encompassed within the scope of the invention are variants, inhibitors and mimetics of B2M which are capable of modulating the role of B2M and thus affecting the pathogenesis of OA.

### BACKGROUND

Osteoarthritis (OA) is a chronic disease in which the articular cartilage that lies on the ends of bones that forms the articulating surface of the joints gradually degenerates over time. There are many factors that are believed to predispose a patient to osteoarthritis including genetic susceptibility, obesity, accidental or athletic trauma, surgery, drugs and heavy physical demands. Osteoarthritis is initiated by damage to the cartilage of joints. The two most common injuries to joints are sports-related injuries and long term “repetitive use” joint injuries. Joints most commonly affected by osteoarthritis are the knees, hips and hands. In most cases, due to the essential weight-bearing function of the knees and hips, osteoarthritis in these joints causes much more disability than osteoarthritis of the hands. As cartilage degeneration progresses, secondary changes occur in other tissues in and around joints including bone, muscle, ligaments, menisci and synovium. The net effect of the primary failure of cartilage tissue and secondary damage to other tissues is that the patient experiences pain, swelling, weakness and loss of functional ability in the afflicted joint(s). These symptoms frequently progress to the point that they have a significant impact in terms of lost productivity and or quality of life consequences for the patient.

Articular cartilage is predominantly composed of chondrocytes, type II collagen, proteoglycans and water. Articular cartilage has no blood or nerve supply and chondrocytes are the only type of cell in this tissue. Chondrocytes are responsible for manufacturing the type II collagen and proteoglycans that form the cartilage matrix. This matrix in turn has physical-chemical properties that allow for saturation of the matrix with water. The net effect of this structural-functional relationship is that articular cartilage has exceptional wear characteristics and allows for almost frictionless movement between the articulating cartilage surfaces. In the absence of osteoarthritis, articular cartilage often provides a lifetime of pain-free weight bearing and unrestricted joint motion even under demanding physical conditions.

During fetal development, articular cartilage is initially derived from the interzone of mesenchymal condensations. The mesenchymal cells cluster together and synthesize matrix proteins. The tissue is recognized as cartilage when the accumulation of matrix separates the cells, which are spherical in shape and are now called chondrocytes. During cartilage formation and growth, chondrocytes proliferate rapidly and synthesize large volumes of matrix. Prior to skeletal maturity, chondrocytes are at their highest level of metabolic activity. As skeletal maturation is reached, the rate of chondrocyte metabolic activity and cell division declines. After completion of skeletal growth, most chondrocytes do not divide but do continue to synthesize matrix proteins such as collagens, proteoglycans and other noncollagenous proteins. (Gussow D, Rein R, Ginjaar I, Hochstenbach F, Seemann G, Kottman A, Ploegh H L. The human beta 2-microglobulin gene. Primary structure and definition of the transcriptional unit. J Immunol 1987;139:3132-8). (Hochman J H, Shimizu Y, DeMars R, Edidin M. Specific associations of fluorescent beta-2 microglobulin with cell surfaces. The affinity of different H-2 and HLA antigens for beta-2-microglobulin. J Immunol 1988;140:2322-9).

Like all living tissues, articular cartilage is continually undergoing a process of renewal in which “old” cells and matrix components are being removed (catabolic activity) and “new” cells and molecules are being produced (anabolic activity). Relative to most tissues, the rate of anabolic/catabolic turnover in articular cartilage is low. Long-term maintenance of the structural integrity of mature cartilage relies on the proper balance between matrix synthesis and degradation. Chondrocytes maintain matrix equilibrium by responding to chemical and mechanical stimuli from their environment. Appropriate and effective chondrocyte responses to these stimuli are essential for cartilage homeostasis. Disruption of homeostasis through either inadequate anabolic activity or excessive catabolic activity can result in cartilage degradation and osteoarthritis. (Bernabeu C, Van de Rijin M, Lerch P G, and Terhorst C. β2-microglobulin from serum associates with MHC class I antigens on the surface of cultured cells. Nature 1984;308:642-5). Most tissues that are damaged and have increased catabolic activity are able to mount an increased anabolic response that allows for tissue healing. Unfortunately, chondrocytes have very limited ability to up-regulate their anabolic activity and increase the synthesis of proteoglycan and type II collagen in response to damage or loss of cartilage matrix. This fundamental limitation of chondrocytes is the core problem that has precluded the development of therapies that can prevent and cure osteoarthritis.

Joint pain is the most common manifestation of early osteoarthritis. The pain tends to be episodic lasting days to weeks and remitting spontaneously. Although redness and swelling of joints is uncommon, joints become tender during a flare-up of osteoarthritis.

“Osteoarthritis” is the most common chronic joint disease. It is characterized by progressive degeneration and eventual loss of cartilage. Currently, there is a need for an effective therapy that will alter the course of osteoarthritis. Further advances in preventing, modifying or curing the osteoarthritic disease process critically depends, at least in part, on a thorough understanding of the molecular mechanisms underlying anabolic and catabolic processes in cartilage.

“Mild” or “early stage osteoarthritis” is difficult to diagnose. The physician relies primarily on the patient's history and physical exam to make the diagnosis of mild osteoarthritis. X-rays do not show the underlying early changes in articular cartilage.

X-ray changes confirm the diagnosis of moderate osteoarthritis. X-rays of normal joints reveal well preserved symmetrical joint spaces. Changes seen on the x-rays of patients with osteoarthritis include new bone formation (osteophytes), joint space narrowing and sclerosis (bone thickening).

The clinical exam of a joint with severe osteoarthritis reveals tenderness, joint deformity and a loss of mobility. Passive joint movement during examination may elicit crepitus or the grinding of bone-on-bone as the joint moves. X-ray changes are often profound: the joint space may be obliterated and misalignment of the joint can be seen. New bone formation (osteophytes) is prominent.

Beta-2 microglobulin (B2M) is a nonglycosylated single polypeptide chain. It is composed of 119 amino acids and has a molecular weight of 11,800 Daltons. (Gussow D, Rein R, Ginjaar I, Hochstenbach F, Seemann G, Kottman A, Ploegh H L. The human beta 2-microglobulin gene. Primary structure and definition of the transcriptional unit. J Immunol 1987;139:3132-8). B2M is synthesized by all nucleated cells and is normally expressed on the cell surface as an integral part of the major histocompatibility class I complex HLA. It can also exist in a non-HLA-associated form. (Hochman J H, Shimizu Y, DeMars R, Edidin M. Specific associations of fluorescent beta-2 microglobulin with cell surfaces. The affinity of different H-2 and HLA antigens for beta-2-microglobulin. J Immunol 1988;140:2322-9). (Bernabeu C, Van de Rijin M, Lerch P G, and Terhorst C. β2-microglobulin from serum associates with MHC class I antigens on the surface of cultured cells. Nature 1984;308:642-5). As HLA molecules are continuously turned over, B2M is shed from the cell membrane into blood and eventually catabolized in the kidney. (Revillard J P, Vincent C. Structure and metabolism of beta-2-microglobulin. Contrib Nephrol 1988;62:44-53). Normally, the serum concentration of B2M is less than 2 mg/litre (or 2 μg/ml) . (Schardijn G H, Statius van Eps L W. Beta 2-microglobulin: its significance in the evaluation of renal function. Kidney Int 1987;32:635-41).

Increased serum levels of B2M have been found in inflammatory and malignant diseases, including rheumatic disorders, infectious diseases and lymphoproliferative disorders. (Rodriguez J, Cortes J, Talpaz M, O'Brien S, Smith T L, Rios M B, Kantarjian H. Serum beta-2 microglobulin levels are a significant prognostic factor in Philadelphia chromosome-positive chronic myelogenous leukemia. Clin Cancer Res 2000;6: 147-52). (Soderblom T, Nyberg P, Pettersson T, Klockars M, Riska H. Pleural fluid beta-2-microglobulin and angiotensin-converting enzyme concentrations in rheumatoid arthritis and tuberculosis. Respiration 1996;63:272-6). (Walters M T, Stevenson F K, Goswami R, Smith J L, Cawley M I Comparison of serum and synovial fluid concentrations of beta 2-microglobulin and C reactive protein in relation to clinical disease activity and synovial inflammation in rheumatoid arthritis. Ann Rheum Dis 1989;48:905-11). Studies have shown that serum levels of B2M can be used as a non-specific marker for certain systemic disease activities and to monitor the effectiveness of therapeutic interventions . (Schardijn G H, Statius van Eps L W. Beta 2-microglobulin: its significance in the evaluation of renal function. Kidney Int 1987;32:635-41). (Betaille R, Durie B G M, Grenier J. Serum beta 2 microglobulin and survival duration in multiple myeloma: a simple reliable marker for staging. Br J Haematol 1983;55:439-447). A significant decrease in the serum levels of B2M was observed after alendronate treatment in rheumatoid arthritis. (Cantatore F P, Acquista C A, Piptone V. Evaluation of bone turnover and osteoclastic cytokines in early rheumatoid arthritis treated with alendronate. J Rheumatol. 1999;26:2318-23). Although B2M has been associated with the immunogenetic system and regarded as a useful marker for monitoring inflammatory and malignant disease activity, its exact function remains unclear.

In addition to its involvement in the immune system, B2M activity has also been associated with osteo-articular tissue in hemodialysis-associated amyloidosis (HAA or B2M amyloidosis) . (Brinckerhoff C E, Mitchell T I, Karmilowicz M J, Kluve-Beckerman B, and Benson M D. Autocrine induction of collagenase by serum amyloid A-like and beta2-microglobulin-like proteins. Sicence 243:655-657, 1989). (Migita K., Eguchi K, Tominaga M, Origuchi T, kawabe Y, and Nagataki S. β2-Microglobulin induces Stromelysin production by human synovial fibroblasts. Bioch. and Biophy. Res. Commu. 1997;239:621-5).

HAA is a complication found in long-term hemodialysis patients. It comprises a wide spectrum of clinical manifestations, including arthritis. (Drueke T B. Dialysis-related amyloidosis. Nephrol Dial Transplant 1998;13 (Suppl 1):58-64).

(Ohashi K. Pathogenesis of beta2-microglobulin amyloidosis. Pathol Int 2001;51:1-10). Amyloid fibrils containing B2M have been demonstrated in joint capsules, synovium, articular cartilage and bone in HAA. (Bindi P, Chanard J. Destructive spondyloarthropathy in dialysis patients: an overview. Nephron 1990;55:104-9).

(Bardin T, Kuntz D, Zingraff J, Voisin M C, Zelmar A, Lansaman J. Synovial amyloidosis in patients undergoing long-term hemodialysis. Arthritis Rheum 1985;28:1052-8).

(Gejyo F, Yamada T, Odani S, Nakagawa Y, Arakawa M, Kunitomo T, Kataoka H, Suzuki M, Hirasawa Y, Shirahama T, et al. A new form of amyloid protein associated with chronic hemodialysis was identified as beta 2-microglobulin. Biochem Biophys Res Commun 1985;129:701-6).

The deposition likely occurs because B2M has high affinity for and binds preferentially to various types of collagen. (Homma N, Gejyo F, Isemura M, Arakawa M. Collagen-binding affinity of beta-2-microglobulin, a preprotein of hemodialysis-associated amyloidosis. Nephron 1989;53:37-40).

The potential for B2M to be involved in cartilage destruction is suggested by studies showing that it induces synthesis of stromelysin (MMP-3) and cyclooxygenase-2 (COX-2) in human synovial fibroblasts. (Migita K., Eguchi K, Tominaga M, Origuchi T, kawabe Y, and Nagataki S. β2-Microglobulin induces Stromelysin production by human synovial fibroblasts. Bioch. and Biophy. Res. Commu. 1997;239:621-5). (Migita K., Tominaga M, Tominaga M, kawabe Y, Aoyagi T, Urayama S, Yamasaki S, Hida A, Kawakmi A, and Eguchi K. Induction of cyclooxygenase-2 in human synovial cells by β2-microglobulin. Kidney International 1999;55:572-8).

Further support derives from a recent study demonstrating that B2M induces matrix metalloproteinase 1 (MMP-1) but not tissue inhibitor of metalloproteinase 1 (TIMP-1) in osteoarthritic synovial fibroblasts. (Moe S M, Singh G K and Bailey A M. beta2-microglobulin induces MMP-1 but not TIMP-1 expression in human synovial fibroblasts. Kidney International 2000;57:2023-34) Thus, B2M appears likely to have a destructive role in amyloidosis-related arthritis. Whether it has a catabolic role in osteoarthritis remains unknown. To date, most studies on B2M have focused on its effects in synovial fibroblasts in non-osteoarthritic diseases. There has been little work exploring its potential effects on chondrocytes or on the pathogenesis of osteoarthritis.

### SUMMARY OF THE INVENTION

The present invention is based upon the surprising discovery that B2M inhibits chondrocyte proliferation and thus its involvement in OA pathogenesis. We have also identified B2M related genes. Encompassed within the scope of our invention are compounds capable of modulating the effect of B2M activity through use of inhibitors, antibodies, variants or mimetics.

In one embodiment, the invention provides for an isolated biomarker comprising two or more genes selected from the group consisting of the 31 genes as set out in Tables 2 and 3.

In one embodiment, the invention provides for an isolated biomarker consisting essentially of the 31 genes as set out in Tables 2 and 3.

In one embodiment, the invention provides for an isolated biomarker comprising one or more polynucleotide sequences from the 5′ region of a gene selected from the group consisting of the 31 genes as set out in Tables 2 and 3.

In one embodiment, the invention provides for an isolated biomarker comprising one or more polynucleotide sequences from the 3′ region of a gene selected from the group consisting of the genes as set out in Tables 2 and 3.

In one embodiment, the invention provides for an isolated biomarker comprising one or more polynucleotide sequences from the internal coding region of a gene selected from the group consisting of the 31 genes as set out in Tables 2 and 3.

In one embodiment, the invention provides for an isolated biomarker comprising the polypeptide sequences encoded by two or more genes selected from the group consisting of the 31 genes as set out in Tables 2 and 3.

In one embodiment, the invention provides for an isolated biomarker consisting essentially of the polypeptide sequences encoded by the 31 genes, as set out in Tables 2 and 3.

In one embodiment, the invention provides for an isolated biomarker comprising the amino terminal polypeptide sequences encoded by one or more polynucleotide sequences from the 5′ region of a gene selected from the group consisting of the 31 genes as set out in Tables 2 and 3.

In one embodiment, the invention provides for an isolated biomarker comprising the carboxy terminal polypeptide sequences encoded by one or more polynucleotide sequences from the 3′ region of a gene selected from the group consisting of the 31 genes as set out in Tables 2 and 3.

In one embodiment, the invention provides for an isolated biomarker comprising the internal polypeptide sequences encoded by one or more polynucleotide sequences from the internal coding region of a gene selected from the group consisting of the 31 genes as set out in Tables 2 and 3.

Another aspect of the invention relates to a method of identifying an inhibitor of B2M activity comprising the steps of contacting chondrocytes with B2M in the presence and absence of a candidate modulator and comparing the proliferation of the chondrocytes in the presence relative to the absence of the candidate modulator, wherein an increase in the proliferation of the chondrocytes in the presence relative to the absence of the candidate modulator identifies the candidate modulator as an inhibitor of B2M activity.

Another aspect of the invention relates to a method of identifying an inhibitor of B2M activity comprising the steps of contacting chondrocytes with B2M in the presence and absence of a candidate modulator and comparing the level of differential expression of a biomarker comprising one or more polynucleotide sequences of one or more genes selected from the group consisting of the 31 genes as set out in Tables 2 and 3 in the presence relative to the absence of the candidate modulator, wherein differentially decreased expression of the biomarker identifies the candidate modulator as an inhibitor of B2M activity.

In one embodiment, the polynucleotide sequences are from the 5′ region of a gene selected from the group consisting of the 31 genes as set out in Tables 2 and 3.

In another embodiment, the polynucleotide sequences are from the 3′ region of a gene selected from the group consisting of the 31 genes as set out in Tables 2 and 3.

In another embodiment, the polynucleotide sequences are from the internal coding region of a gene selected from the group consisting of the 31 genes as set out in Tables 2 and 3.

Another aspect of the invention relates to a method of identifying an inhibitor of B2M activity comprising the steps of contacting chondrocytes with B2M in the presence and absence of a candidate modulator, and comparing the level of differential expression of a biomarker comprising one or more polypeptide sequences of one or more genes selected from the group consisting of the 31 genes as set out in Tables 2 and 3 in the presence relative to the absence of the candidate modulator, wherein differentially increased expression of the biomarker identifies the candidate modulator as an inhibitor of B2M or B2M related activity.

In one embodiment, the polypeptide sequences are amino terminal polypeptide sequences encoded by one or more polynucleotide sequences from the 5′ region of a gene selected from the group consisting of the 31 genes as set out in Tables 2 and 3.

In another embodiment, the polypeptide sequences are carboxy terminal polypeptide sequences encoded by one or more polynucleotide sequences from the 3′ region of a gene selected from the group consisting of the 31 genes as set out in Tables 2 and 3.

In another embodiment, the polypeptide sequences are internal polypeptide sequences encoded by one or more polynucleotide sequences from the internal coding region of a gene selected from the group consisting of the 31 genes as set out in Tables 2 and 3.

In yet another embodiment, the invention further provides for a composition comprising two or more probes that specifically hybridize to an isolated biomarker comprising two or more genes selected from the group consisting of the 31 genes as set out in Tables 2 and 3.

In a further embodiment, the probes are single or double stranded RNA or single or double stranded DNA.

In one embodiment, the invention provides for a composition comprising a ligand that specifically binds to a polypeptide encoded by a gene of an isolated biomarker comprising the polypeptide sequences encoded by two or more genes selected from the group consisting of the 31 genes as set out in Tables 2 and 3.

In another embodiment, the invention provides for a ligand that is a monoclonal antibody.

In another embodiment, the invention provides for a kit comprising an isolated biomarker of one or more of the subject isolated biomarkers described above and packaging means therefore.

In another embodiment, the invention provides for a microarray comprising an isolated biomarker of one or more of the subject isolated biomarkers, described above, bound to a solid support.

In another embodiment, the invention provides for a microarray comprising ligands bound to a support, where the ligands specifically bind to one or more of the subject isolated biomarkers, described above.

### BRIEF DESCRIPTION OF THE DRAWINGS

The objects and features of the invention can be better understood with reference to the following detailed description and drawings.

Table 1 is a chart, according to one embodiment of the invention, showing the levels of B2M in synovial fluid as determined by ELISA. Synovial fluid isolated from patients identified as normal, or having either mild, moderate, marked or severe OA were tested for the level of B2M.

Table 2 is a chart, according to one embodiment of the invention, listing B2M related genes which have been identified on the basis of the up regulation of these genes in response to treatment of chondrocytes with B2M.

Table 3 is a chart, according to one embodiment of the invention, listing B2M related genes which have been identified on the basis of the down-regulation of these genes in response to treatment of chondrocytes with B2M.

FIG. 1a , is an autoradiograph, in one embodiment of the invention, demonstrating the level of B2M mRNA expression in human fetal, mild and severe OA cartilage as determined by Reverse Transcription PCR (RT-PCR). Pooled samples from fetal, mild and severe OA cartilage were used to extract total RNA. 1 μg RNA was then used for reverse transcription and PCR amplification for B2M and GAPDH, which gives 415 bp and 370 bp size PCR product respectively. M=mild OA, S=severe OA cartilage, and F=fetal cartilage.

FIG. 1b, in one embodiment of the invention, is a bar graph demonstrating relative ESTs frequency levels of B2M in fetal, normal adult, mild and severe OA cartilage cDNA libraries. B2M EST copy number in each library was divided into the total EST number of the corresponding library: fetal=6/13398=0.04%, normal adult=88/17151=0.51%, mild OA=200/12651=1.58%, severe OA=196/14222=1.38%.

FIG. 2, in one embodiment of the invention, is a bar graph demonstrating the B2M levels in normal and OA synovial fluid as detected by ELISA. A total of 55 synovial fluid samples were tested using a B2M enzyme immunoassay test kit. Ten microlitres of each sample were measured in duplicate. B2M concentration was calculated based on the standard curve. The average of B2M concentrations in normal (nor, 9 samples), mild (mioa, 11 samples), moderate (mooa, 10 samples), marked (maoa, 16 samples) and severe (seoa, 9 samples) was compared. Label * means that there is a significant difference in B2M level compared to normal (p<0.05).

FIG. 3, in one embodiment of the invention, is a bar graph demonstrating the B2M levels in cartilage organ cultured medium. Severe OA cartilage slices were cultured in a 24-well plate at one piece/well. 10 μl of medium was collected at 24 hr, 48 hr and 74 hr culture intervals and tested for cumulative release of B2M by ELISA. The mean and standard deviation of three experiments were shown (24 hr: 1.03±0.35, 48 hr: 1.42±0.37, 72 hr: 2.03±0.9). Student t-test results were: 24 hr/48 hr, p=0.359; 24 hr/72 hr, p=0.051; 48 hr/72 hr, p=0.089.

FIG. 4, in one embodiment of the invention, is a bar graph demonstrating the effect of B2M on human severe OA chondrocyte proliferation. Chondrocytes were seeded at 1×10⁴ cells/well in triplicate in a 96-well plate. Cells were cultured with or without FCS at various concentrations of B2M (0, 0.1, 1.0, and 10.0 μg/ml) for 48 hr. Then 10 μl of WST-1 (a tetrazolium salt that can be cleaved to formazan by mitochondria dehydrogenases in live cells) was added to each well and the plate was scanned by a microplate autoreader at an absorbance of 450 nm. One of the three experiments with three different severe OA donors was shown here. Label * means that there is a significant difference between treated and non-treated samples (0 μg/ml B2M) (p<0.05).

FIG. 5, in one embodiment of the invention, is a Scatter plot of fluorescent signal intensity from the hybridization using B2M treated or non-treated OA chondrocytes as a probe to the microarray. The scatter plot was generated using Sigma Plot 5.0. The middle line represents a slope of one after normalization. The other two lines represent two-fold differential expression in channel 1 (Ch1 Cy3) and channel 2 (Ch2 Cy5).

### DETAILED DESCRIPTION

The practice of the present invention will employ, unless otherwise indicated, conventional techniques of molecular biology, microbiology and recombinant DNA techniques, which are within the skill of the art. Such techniques are explained fully in the literature. See, e.g., Sambrook, Fritsch & Maniatis, 1989, Molecular Cloning: A Laboratory Manual, Second Edition; Oligonucleotide Synthesis (M. J. Gait, ed., 1984); Nucleic Acid Hybridization (B. D. Harnes & S. J. Higgins, eds., 1984); A Practical Guide to Molecular Cloning (B. Perbal, 1984); and a series, Methods in Enzymology (Academic Press, Inc.); Short Protocols In Molecular Biology, (Ausubel et al., ed., 1995).

Definitions

The following definitions are provided for specific terms which are used in the following written description.

As used herein, the term “biomarker” refers to a set of genes that are differentially regulated in chondrocytes in the presence of B2M.

As used herein, “isolated biomarker” means that the biomarker is isolated from and therefore not part of a mixture containing a set of OA genes including those taught in WO 02/070737, of more than 50 genes.

As used herein, “consisting essentially of” refers to the maximum number of genes that are required for the use of a biomarker to identify B2M activity in chondrocytes. In one embodiment, a biomarker of B2M activity consists essentially of at least the 31 genes disclosed in Tables 2 and 3 combined.

A “gene”, as used herein, refers to DNA encoding mRNA and does not include promoters and enhancers upstream of the coding region.

As used herein, “polypeptide sequences encoded by” refers to the amino acid sequences obtained after translation of the protein coding region of a gene, as defined herein. The mRNA nucleotide sequence for each of the genes in Tables 2 and 3 is identified by its Genbank or Unigene Accession numbers, where available, and the corresponding polypeptide sequence is identified by a Protein Accession number, where available. The Genbank Accession numbers identified in Tables 2 and 3 may provide the location of the 5′ UTR, protein coding region (CDS) and 3′ UTR within the mRNA nucleotide sequence of each of the genes.

As used herein, the “5′ end” refers to the end of an mRNA up to the first 1000 nucleotides or ⅓ of the mRNA (where the full length of the mRNA does not include the poly A tail), starting at the first nucleotide of the mRNA. The “5′ region” of a gene refers to a polynucleotide (double-stranded or single-stranded) located within or at the 5′ end of a gene, and includes, but is not limited to, the 5′ untranslated region, if that is present, and the 5′ protein coding region of a gene. The 5′ region is not shorter than 8 nucleotides in length and not longer than 1000 nucleotides in length. Other possible lengths of the 5′ region include but are not limited to 10, 20, 25, 50, 100, 200, 400, and 500 nucleotides.

As used herein, the “3′ end” refers to the end of an mRNA up to the last 1000 nucleotides or ⅓ of the mRNA, where the 3′ terminal nucleotide is that terminal nucleotide of the coding or untranslated region that adjoins the poly-A tail, if one is present. That is, the 3′ end of an mRNA does not include the poly-A tail, if one is present. The “3′ region” of a gene refers to a polynucleotide (double-stranded or single-stranded) located within or at the 3′ end of a gene, and includes, but is not limited to, the 3′ untranslated region, if that is present, and the 3′ protein coding region of a gene. The 3′ region is not shorter than 8 nucleotides in length and not longer than 1000 nucleotides in length. Other possible lengths of the 3′ region include but are not limited to 10, 20, 25, 50, 100, 200, 400, and 500 nucleotides.

As used herein, the “internal coding region” of a gene refers to a polynucleotide (double-stranded or single-stranded) located between the 5′ region and the 3′ region of a gene as defined herein. The “internal coding region” is not shorter than 8 nucleotides in length and not longer than 1000 nucleotides in length. Other possible lengths of the “internal coding region” include but are not limited to 10, 20, 25, 50, 100, 200, 400, and 500 nucleotides.

The 5′, 3′ and internal regions are non-overlapping and may, but need not be contiguous, and may, but need not, add up to the full length of the corresponding gene.

As used herein, the “amino terminal” region of a polypeptide refers to the polypeptide sequences encoded by polynucleotide sequences (double-stranded or single-stranded) located within or at the 5′ end of a gene, and includes, but is not limited to, the 5′ protein coding region of a gene. As used herein, the “amino terminal” region refers to the amino terminal end of a polypeptide up to the first 300 amino acids or ⅓ of the polypeptide, starting at the first amino acid of the polypeptide. The “amino terminal” region of a polypeptide is not shorter than 3 amino acids in length and not longer than 350 amino acids in length. Other possible lengths of the “amino terminal” region of a polypeptide include but are not limited to 5, 10, 20, 25, 50, 100 and 200 amino acids.

As used herein, the “carboxy terminal” region of a polypeptide refers to the polypeptide sequences encoded by polynucleotide sequences (double-stranded or single-stranded) located within or at the 3′ end of a gene, and includes, but is not limited to, the 3′ protein coding region of a gene. As used herein, the “carboxy terminal” region refers to the carboxy terminal end of a polypeptide up to 300 amino acids or ⅓ of the polypeptide from the last amino acid of the polypeptide. The “3′ end” does not include the polyA tail, if one is present. The “carboxy terminal” region of a polypeptide is not shorter than 3 amino acids in length and not longer than 350 amino acids in length. Other possible lengths of the “carboxy terminal” region of a polypeptide include, but are not limited to, 5, 10, 20, 25, 50, 100 and 200 amino acids.

As used herein, the “internal polypeptide region” of a polypeptide refers to the polypeptide sequences located between the amino terminal region and the carboxy terminal region of a polypeptide, as defined herein. The “internal polypeptide region” of a polypeptide is not shorter than 3 amino acids in length and not longer than 350 amino acids in length. Other possible lengths of the “internal polypeptide region” of a polypeptide include, but are not limited to, 5, 10, 20, 25, 50, 100 and 200 amino acids.

The amino terminal, carboxy terminal and internal polypeptide regions of a polypeptide are non-overlapping and may, but need not be contiguous, and may, but need not, add up to the full length of the corresponding polypeptide.

An “mRNA” means an RNA complimentary to a gene; an mRNA includes a protein coding region and also may include 5′ end and 3′ untranslated regions (UTR).

A “coding region” refers to a DNA encoding mRNA.

A “protein coding region” refers to the portion of the mRNA encoding a polypeptide.

As used herein, “inhibitor” refers to any compound that reduces the activity of B2M by 1%, 5%, 10%, 20%, 30%, 40%, 50%, 60%, 70%, 80%, 90% or more. In a preferred embodiment, an “inhibitor” of the present invention includes, but is not limited to, antibodies, mimetics, variants, antisense molecules, ribozymes and RNAi molecules as defined herein.

As used herein, the term “amplified”, when applied to a nucleic acid sequence, refers to a process whereby one or more copies of a particular nucleic acid sequence is generated from a template nucleic acid, preferably by the method of polymerase chain reaction (Mullis and Faloona, 1987, Methods Enzymol., 155:335). “Polymerase chain reaction” or “PCR” refers to an in vitro method for amplifying a specific nucleic acid template sequence. The PCR reaction involves a repetitive series of temperature cycles and is typically performed in a volume of 50-100 μl. The reaction mix comprises dNTPs (each of the four deoxynucleotides dATP, dCTP, dGTP, and dTTP), primers, buffers, DNA polymerase, and nucleic acid template. The PCR reaction comprises providing a set of polynucleotide primers wherein a first primer contains a sequence complementary to a region in one strand of the nucleic acid template sequence and primes the synthesis of a complementary DNA strand, and a second primer contains a sequence complementary to a region in a second strand of the target nucleic acid sequence and primes the synthesis of a complementary DNA strand, and amplifying the nucleic acid template sequence employing a nucleic acid polymerase as a template-dependent polymerizing agent under conditions which are permissive for PCR cycling steps of (i) annealing of primers required for amplification to a target nucleic acid sequence contained within the template sequence, (ii) extending the primers wherein the nucleic acid polymerase synthesizes a primer extension product. “A set of polynucleotide primers” or “a set of PCR primers” can comprise two, three, four or more primers. In one embodiment, an exo-Pfu DNA polymerase is used to amplify a nucleic acid template in PCR reaction. Other methods of amplification include, but are not limited to, ligase chain reaction (LCR), polynucleotide-specific based amplification (NSBA), or any other method known in the art.

As used herein, the term “differential expression” refers to a difference in the level of expression of a gene, as measured by the amount or level of RNA, including mRNA, complementary to the gene, in one sample as compared with the level of expression of the same gene in a second sample. Differential expression can be determined as a result of differential hybridization or through other known methods in the art used to measure the level or amount of mRNA expression.

As used herein the term “differential expression” also refers to a difference in the level of expression of a gene, as measured by the amount or level of protein encoded by the gene, in one sample as compared with the amount or level of protein expression of the same gene in a second sample. Differential protein expression can be determined as a result of binding to monoclonal antibodies that are specific for the particular protein or through other known methods in the art used to measure the level or amount of protein expression.

“Differentially increased expression” refers to 1.1 fold, 1.2 fold, 1.4 fold, 1.6 fold, 1.8 fold or more. “Differentially decreased expression” refers to less than 1.0 fold, 0.8 fold, 0.6 fold, 0.4 fold, 0.2 fold, 0.1 fold or less.

As used herein, the term “control” in the context of this invention refers to chondrocytes that were cultured under standard conditions in the absence of additional components.

As used herein, a “ligand” is a molecule that specifically binds to a polypeptide encoded by one of the genes of the invention. A ligand can be a nucleic acid (RNA or DNA), polypeptide, peptide or chemical compound. A ligand of the invention can be a peptide ligand, e.g., a scaffold peptide, a linear peptide, or a cyclic peptide. In a preferred embodiment, the polypeptide ligand is an antibody. The antibody can be a human antibody, a chimeric antibody, a recombinant antibody, a humanized antibody, a monoclonal antibody, or a polyclonal antibody. The antibody can be an intact immunoglobulin, e.g., an IgA, IgG, IgE, IgD, IgM or subtypes thereof. The antibody can be conjugated to a functional moiety (e.g., a compound which has a biological or chemical function (which may be a second different polypeptide, a therapeutic drug, a cytotoxic agent, a detectable moiety, or a solid support. A polypeptide ligand e.g. antibody of the invention interacts with a polypeptide, encoded by one of the genes of a biomarker, with high affinity and specificity. For example, the polypeptide ligand binds to a polypeptide, encoded by one of the genes of a biomarker, with an affinity constant of at least 10⁷ M⁻¹, preferably, at least 10⁸ M⁻¹, 10⁹ M⁻¹, or 10¹⁰ M⁻¹.

In a preferred embodiment, “antibodies” refer to neutralizing antibodies or antibodies which are capable of modulating the effect of B2M or the gene products of the B2M related genes and includes fragments thereof. The term “antibodies” is also intended to include antibodies to receptors specific for one or more of B2M or the gene products of the B2M related genes. Antibodies can be fragmented using conventional techniques and the fragments screened for utility in the same manner as described above.

As used herein, the term “specifically binds” refers to the interaction of two molecules, e.g., a ligand and a protein or peptide, wherein the interaction is dependent upon the presence of particular structures on the respective molecules. For example, when the two molecules are protein molecules, a structure on the first molecule recognizes and binds to a structure on the second molecule, rather than to proteins in general. “Specific binding”, as the term is used herein, means that a molecule binds its specific binding partner with at least 2-fold greater affinity, and preferably at least 10-fold, 20-fold, 50-fold, 100-fold or higher affinity than it binds a non-specific molecule.

As used herein, the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 Kd or 214 amino acids) are encoded by a variable region gene at the NH2-terminus (about 110 amino acids) and a kappa or lambda constant region gene at the COOH-terminus. Full-length immunoglobulin “heavy chains” (about 50 Kd or 446 amino acids), are similarly encoded by a variable region gene (about 116 amino acids) and one of the other aforementioned constant region genes, e.g., gamma (encoding about 330 amino acids).

The term “antibody” also encompasses antigen-binding fragments of an antibody. The term “antigen-binding fragment” of an antibody (or simply “antibody portion,” or “fragment”), as used herein, refers to one or more fragments of a full-length antibody that retain the ability to specifically bind to a polypeptide encoded by one of the genes of a biomarker of the invention. Examples of binding fragments encompassed within the term “antigen-binding fragment” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al, (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies. The antibody is preferably monospecific, e.g., a monoclonal antibody, or antigen-binding fragment thereof. The term “monospecific antibody” refers to an antibody that displays a single binding specificity and affinity for a particular target, e.g., epitope. This term includes a “monoclonal antibody” or “monoclonal antibody composition,” which as used herein refer to a preparation of antibodies or fragments thereof of single molecular composition.

As used herein “activator” is meant a substance or a group of substances having the ability to increase (e.g., by at least 10%, or 25% or 50% or more compared to a standard) the activity of B2M or the product(s) of the B2M related genes.

By the term “antisense molecule” is meant a nucleotide sequence which can hybridize to the mRNA corresponding to B2M or a B2M related gene and modulate the level of B2M or the products of the B2M related genes. Antisense nucleic acids can be at least six nucleotides in length, and are preferably less that about 100 and more preferably less than about 50, 25, 17 or 10 nucleotides in length.

As used herein, “attaching” or “spotting” refers to a process of depositing a nucleic acid onto a solid substrate to form a nucleic acid array such that the nucleic acid is irreversibly bound to the solid substrate via covalent bonds, hydrogen bonds or ionic interactions.

As used herein, “B2M related genes” refers to genes which are up regulated or down regulated as a result of incubation with B2M.

As used herein, “cartilage” or “articular cartilage” refers to elastic, translucent connective tissue in mammals, including human and other species. Cartilage is composed predominantly of chondrocytes, type II collagen, small amounts of other collagen types, other noncollagenous proteins, proteoglycans and water, and is usually surrounded by a perichondrium, made up of fibroblasts, in a matrix of type I and type II collagen as well as other proteoglycans. Although most cartilage becomes bone upon maturation, some cartilage remains in its original form in locations such as the nose, ears, knees, and other joints. The cartilage has no blood or nerve supply and chondrocytes are the only type of cell in this tissue.

As used herein, a “cartilage nucleic acid sample”, refers to nucleic acids derived from cartilage. Preferably, a cartilage nucleic acid sample is RNA or is a nucleic acid corresponding to RNA, for example, cDNA.

As used herein, “chondrocyte” refers to cells isolated from cartilage.

As used herein, the term “down regulated” or “down regulation” refers to differential expression wherein the RNA, including mRNA in a first sample is expressed in less amounts as compared with a second sample and includes decreased differential expression of 1.5 fold, 2 fold, 2.5 fold, 3 fold etc.

The term “dosing”, as used herein, refers to the administration of a substance (e.g. B2M and/or one or more of the products of the B2M related genes, as well as activators, variants, inhibitors, mimetics, antibodies and antisense molecules of B2M or the products of the B2M related genes) to achieve a therapeutic objective (e.g. the treatment of a OA or the modification of chondrocyte proliferation).

As used herein, “fetal” cartilage samples refer to samples taken from a fetus. The chondrocytes of fetal cartilage have a higher level of metabolic activity and cell division rates as compared to chondrocytes from cartilage derived from either a normal adult or from an adult diagnosed with any stage of OA (mild, moderate, marked and severe).

By the terms “functionally equivalent variant” or “variant” is meant minor modifications to the gene products described herein, and may include replacement of one or more amino acids with one or more amino acid substitutions, insertions, and/or deletions. Amino acid substitutions may be of a conserved nature or may be non-conserved. Conserved amino acid substitutions involve replacing one or more amino acids of the proteins of the invention with amino acids of similar charge, size, and/or hydrophobicity characteristics. When non-conserved substitutions are made the resulting analog should be functionally equivalent. Non-conserved substitutions involve replacing one or more amino acids which possess dissimilar charge, size, and/or hydrophobicity characteristics. Variants also include post translational modifications to the gene products, including enzymatic and non-enzymatic modifications, including glycosylation, glycation, hydroxylation and the like. The term “variant” also encompasses minor variations as described above to the mimetics and inhibitors of the invention.

As used herein, the term “hybridizing to” or “hybridization” refers to the hydrogen binding with a complementary nucleic acid, via an interaction between for example, a target nucleic acid sequence and a nucleic acid member in an array.

As used herein, by the term “inhibitor” or “inhibitor molecule” is meant a molecule or a group of molecules having the ability to reduce the activity of B2M or the product(s) of the B2M related genes. A molecule is said to be an “inhibitor” if it reduces the activity of B2M or the product(s) of the B2M related genes by at least 10%, more preferably, at least 20%, or 25%, or 50% or more, compared to a standard.

As used herein, “isolated” or “purified” when used in reference to a nucleic acid means that a naturally occurring sequence has been removed from its normal cellular (e.g., chromosomal) environment or is synthesized in a non-natural environment (e.g., artificially synthesized). Thus, an “isolated” or “purified” sequence may be in a cell-free solution or placed in a different cellular environment. The term “purified” does not imply that the sequence is the only nucleotide present, but that it is essentially free (about 90-95% pure) of non-nucleotide material naturally associated with it, and thus is distinguished from isolated chromosomes.

As used herein, the term “level of expression” refers to the measurable expression level of a given nucleic acid. The level of expression of a nucleic acid is determined by methods well known in the art. The term “differentially expressed” or “changes in the level of expression” refers to an increase or decrease in the measurable expression level of RNA including mRNA complementary to a gene in one sample as compared with the level of expression of the same gene in a second sample. Differential expression can be determined as a result of differential hybridization, for example via microarray analysis, or through other known methods in the art used to measure the level or amount of mRNA. As used herein, “differentially expressed” when referring to microarray analysis means the ratio of the level of expression of a given polynucleotide in one sample and the expression level of the given polynucleotide in another sample is not equal to 1.0. “Differentially expressed” when referring to microarray analysis according to the invention also means the ratio of the expression level of a given polynucleotide in one sample and the expression level of the given polynucleotide in another sample where the ratio is greater than or less than 1.0 and includes greater than 1.5 and less than 0.7, as well as greater than 2.0 and less than 0.5. A nucleic acid also is said to be differentially expressed in two samples if one of the two samples contains no detectable expression of the nucleic acid. Absolute quantification of the level of expression of a nucleic acid can be accomplished by including known concentration(s) of one or more control nucleic acid species, generating a standard curve based on the amount of the control nucleic acid and extrapolating the expression level of the “unknown” nucleic acid species from the hybridization intensities of the unknown with respect to the standard curve. The level of expression can be measured by hybridization analysis using labeled target nucleic acids according to methods well known in the art. The label on the target nucleic acid can be a luminescent label, an enzymatic label, a radioactive label, a chemical label or a physical label. Preferably, target nucleic acids are labeled with a fluorescent molecule. Preferred fluorescent labels include, but are not limited to: fluorescein, amino coumarin acetic acid, tetramethylrhodamine isothiocyanate (TRITC), Texas Red, Cy3 and Cy5.

By the term “mimetic” is meant a substance that mimics B2M or one or more of the functional epitopes of B2M or one or more of the products of the B2M related genes or one or more of the epitopes of the products of the B2M related genes of the present invention so as to increase the proliferation of chondrocytes and/or modulate the pathogenesis of OA.

As used herein, “modulation of activity” is meant the ability to increase or decrease (e.g., at least by 10% or 25% or 50% or more compared to a standard) the amount or rate of transcription or degradation of mRNA corresponding to B2M or the B2M related genes or increase or decrease the rate or amount of translation or the amount of protein turnover of B2M or the product(s) of the B2M related genes. The activity of B2M or the B2M related genes or gene products may be modulated by increasing or decreasing the function or expression of B2M or the B2M related genes or gene products.

As used herein, the term “candidate modulator” refers to a molecule that is capable of modulating (i.e., increasing or decreasing) the activity of B2M or the B2M related genes or gene products. The decrease or increase of B2M or the B2M related genes or gene products in a sample contacted with a candidate modulator is usually measured against a standard, which for example, can be a sample not contacted with the same candidate. The sample may be a control protein, e.g., albumin; or a control cell.

As used herein, “mRNA integrity” refers to the quality of mRNA extracts from cartilage samples. mRNA extracts with good integrity do not appear to be degraded when examined by methods well known in the art, for example, by RNA agarose gel electrophoresis (e.g., Ausubel et al., John Weley & Sons, Inc., 1997, Current Protocols in Molecular Biology). Preferably, the mRNA samples have good integrity (e.g., less than 10%, preferably, less than 5%, and more preferably, less than 1% of the mRNA is degraded) to truly represent the gene expression levels of the cartilage samples from which they are extracted.

As used herein, “normal” refers to an individual who has not shown any OA symptoms or has not been diagnosed with cartilage injury or OA. “Normal”, according to the invention, also refers to a sample taken from a normal individual within 14 hours post-mortem. A normal cartilage tissue sample, for example, refers to the whole or a piece of cartilage isolated from cartilage tissue within 14 hours post-mortem from an individual who was not diagnosed with OA and whose corpse does not show any symptoms of OA at the time of tissue removal. In alternative embodiments of the invention, the “normal” cartilage tissue sample is isolated from cartilage tissue less than 14 hours post-mortem, e.g., within 13 hours, 12 hours, 11 hours, 10 hours, 9 hours, 8 hours, 7 hours, 6 hours, 5 hours, 4 hours, 3 hours, 2 hours, or 1 hour post-mortem. In one embodiment of the invention, the “normal” cartilage sample is isolated at 14 hours post-mortem and the integrity of mRNA samples extracted is confirmed.

As used herein, a “nucleic acid target” or “a target nucleic acid” is defined as a nucleic acid capable of binding to a nucleic acid member of complementary sequence through one or more types of chemical bonds, usually through complementary base pairing, i.e., through hydrogen bond formation. As used herein, a nucleic acid target may include natural (i. e., A, G, C, or T) or modified bases (7-deazaguanosine, inosine, etc.). In addition, the bases in nucleic acid probe may be joined by a linkage other than a phosphodiester bond, so long as it does not interfere with hybridization (i.e., the probe still specifically binds to its complementary sequence under standard stringent or selective hybridization conditions). Thus, nucleic acid targets may be peptide nucleic acids in which the constituent bases are joined by peptide bonds rather than phosphodiester linkages. Preferably, the nucleic acid targets are derived from human cartilage, blood or synovial fluid extracts. More preferably, the nucleic acid targets are single- or double-stranded DNA, RNA, or DNA-RNA hybrids, from human cartilage, blood or synovial fluid RNA extracts, and preferably from mRNA extracts.

As defined herein, a “nucleic acid array” refers a plurality of unique nucleic acids (or “nucleic acid members”) attached to one surface of a solid support at a density exceeding 20 different nucleic acids/cm2 wherein each of the nucleic acid members is attached to the surface of the solid support in a non-identical pre-selected region. In one embodiment, the nucleic acid member attached to the surface of the solid support is DNA. In a preferred embodiment, the nucleic acid member attached to the surface of the solid support is cDNA. In another preferred embodiment, the nucleic acid member attached to the surface of the solid support is cDNA synthesized by polymerase chain reaction (PCR). Preferably, a nucleic acid member of the array according to the invention is at least 50 nucleotides in length. Preferably, a nucleic acid member of the array is less than 6,000 nucleotides in length. More preferably, a nucleic acid member of the array comprises an array less than 500 nucleotides in length. In one embodiment, the array comprises at least 500 different nucleic acid members attached to one surface of the solid support. In another embodiment, the array comprises at least 10 different nucleic acid members attached to one surface of the solid support. In yet another embodiment, the array comprises at least 10,000 different nucleic acid members attached to one surface of the solid support. In yet another embodiment, the array comprises at least 15,000 different nucleic acid members attached to one surface of the solid support.

The term “nucleic acid”, as used herein, is interchangeable with the term “polynucleotide”.

As used herein, “osteoarthritis” refers to a chronic disease in which the articular cartilage that lies on the ends of bones that form the articulating surface of the joints gradually degenerates over time. Cartilage degeneration can be caused by an imbalanced catabolic activity (removal of “old” cells and matrix components) and anabolic activity (production of “new” cells and molecules) (Westacott et al., 1996, Semin Arthritis Rheum, 25:254-72).

As used herein, the term “osteoarthritis (OA) staging” or “osteoarthritis (OA) grading” refers to determining the degree of advancement or progression of the disease in the cartilage. In order to classify cartilage into different disease stages, a scoring system is used according to known methods in the art. Preferably the scoring system described in Marshall (Marshall W., 1996, The Journal of Rheumatology, 23:582-584, incorporated by reference) is used. According to this method, each of the 6 articular surfaces (patella, femoral trochlea, medial femoral condyle, medial tibial plateau, lateral femoral condyle and lateral tibial plateau) is assigned a cartilage grade based on the worst lesion present on that specific surface. A scoring system is then applied in which each articular surface receives an OA severity number value that reflects the cartilage severity grade for that surface. For example, if the medial femoral condyle has a grade I lesion as its most severe cartilage damage a value of 1 is assigned. A total score for the patient is then derived from the sum of the scores on the 6 articular surfaces. Based on the total score, each patient is placed into one of 4 OA groups: mild (early) (1-6), moderate (7-12), marked (13-18) and severe (>18).

As used herein, the term “pathogenesis of Osteoarthritis (OA)” refers to the progression of osteoarthritis as determined by “osteoarthritis (OA) staging” or “osteoarthritis (OA) grading” as defined herein.

As used herein, the term “modulation of the pathogenesis of osteoarthritis” means the altering of the progression of the disease so as to reduce or reverse the symptomatic and or physical effects of osteoarthritis and also includes the apparent reduction in osteoarthritis as determined by osteoarthritis staging or osteoarthritis grading.

As used herein, “patient” refers to a mammal who is diagnosed with a mild, moderate, marked, or severe form of OA.

As used herein, “products of B2M related genes” refers to the protein produced from the B2M related genes.

As used herein, “polynucleotide(s)”, which includes “nucleic acid(s)” “nucleic acid sequences”, “sequences” and “Express Sequence Tags”(EST(s)), generally refers to any polyribonucleotide or poly-deoxyribonucleotide, which may be unmodified RNA or DNA or modified RNA or DNA. “Polynucleotides” include, without limitation, single- and double-stranded nucleic acids. As used herein, the term “polynucleotide(s)” also includes DNAs or RNAs as described above, that contain one or more modified bases. Thus, DNAs or RNAs with backbones modified for stability or for other reasons are “polynucleotides”. The term “polynucleotides” as it is used herein embraces such chemically, enzymatically or metabolically modified forms of polynucleotides, as well as the chemical forms of DNA and RNA characteristic of viruses and cells, including for example, simple and complex cells.

As used herein, the term “probe” refers to an oligonucleotide which forms a duplex structure with a sequence in the target nucleic acid, due to complementarity of at least one sequence in the probe with a sequence in the target region. A probe is at least 8 nucleotides in length and less than the length of a complete gene. A probe may be 10, 20, 30, 50, 75, 100, 150, 200, 250, 400, 500 and up to 2000 nucleotides in length as long as it is less the full length of the target gene.

As used herein, “a plurality of” or “a set of” refers to more than two, for example, 3 or more, 100 or more, or 1000 or more, or 10,000 or more.

As used herein, “pre-selected region”, “predefined region”, or “unique position” refers to a localized area on a substrate which is, was, or is intended to be used for the deposit of a nucleic acid and is otherwise referred to herein in the alternative as a “selected region” or simply a “region.” The pre-selected region may have any convenient shape, e.g., circular, rectangular, elliptical, wedge-shaped, etc. In some embodiments, a pre-selected region is smaller than about 1 cm2, more preferably less than 1 mm2, still more preferably less than 0.5 mm2, and in some embodiments less than 0.1 mm2. A nucleic acid member at a “pre-selected region”, “predefined region”, or “unique position” is one whose identity (e.g., sequence) can be determined by virtue of its position at the region or unique position.

As used herein, “stably associated” refers to a nucleic acid that is irreversibly bound to a solid substrate to form an array via covalent bonds, hydrogen bonds or ionic interactions such that the nucleic acid retains its unique pre-selected position relative to all other nucleic acids that are stably associated with an array, or to all other pre-selected regions on the solid substrate under conditions in which an array is typically analyzed (i.e., during one or more steps of hybridization, washes, and/or scanning, etc.).

As used herein, “solid substrate” or “solid support” refers to a material having a rigid or semi-rigid surface. The terms “substrate” and “support” are used interchangeably herein with the terms “solid substrate” and “solid support”. The solid support may be biological, non-biological, organic, inorganic, or a combination of any of these, existing as particles, strands, precipitates, gels, sheets, tubing, spheres, beads, containers, capillaries, pads, slices, films, plates, slides, chips, etc. Often, the substrate is a silicon or glass surface, (poly)tetrafluoroethylene, (poly)vinylidendifluoride, polystyrene, polycarbonate, a charged membrane, such as nylon 66 or nitrocellulose, or combinations thereof. In a preferred embodiment, the solid support is glass. Preferably, at least one surface of the substrate will be substantially flat. Preferably, the surface of the solid support will contain reactive groups, including, but not limited to, carboxyl, amino, hydroxyl, thiol, and the like. In one embodiment, the surface is optically transparent.

As herein used, the term “standard stringent conditions” means hybridization will occur only if there is at least 95% and preferably, at least 97% identity between the sequences, wherein the region of identity comprises at least 10 nucleotides. In one embodiment, the sequences hybridize under stringent conditions following incubation of the sequences overnight at 42° C., followed by stringent washes (0.2×SSC at 65° C). As several factors affect the stringency of hybridization, the combination of parameters is more important than the absolute measure of a single factor.

As used herein, “synovial fluid” refers to fluid secreted from the “synovial sac” which surrounds each joint. Synovial fluid serves to protect the joint, lubricate the joint and provide nourishment to the articular cartilage. Synovial fluid useful according to the invention contains cells from which RNA can be isolated according to methods well known in the art as described herein.

As used herein, “specific hybridization” or “selective hybridization” refers to hybridization which occurs when two nucleic acid sequences are substantially complementary (at least about 65% complementary over a stretch of at least 14 to 25 nucleotides, preferably at least about 75%, more preferably at least about 90% complementary). See Kanehisa, M., 1984, Nucleic acids Res., 12:203, incorporated herein by reference. As a result, it is expected that a certain degree of mismatch is tolerated. Such mismatch may be small, such as a mono-, di- or tri-nucleotide. Alternatively, a region of mismatch can encompass loops, which are defined as regions in which there exists a mismatch in an uninterrupted series of four or more nucleotides. Numerous factors influence the efficiency and selectivity of hybridization of two nucleic acids, for example, a nucleic acid member on a array, to a target nucleic acid sequence. These factors include nucleic acid member length, nucleotide sequence and/or composition, hybridization temperature, buffer composition and potential for steric hindrance in the region to which the nucleic acid member is required to hybridize. A positive correlation exists between the nucleic acid member length and both the efficiency and accuracy with which a nucleic acid member will anneal to a target sequence. In particular, longer sequences have a higher melting temperature (TM) than do shorter ones, and are less likely to be repeated within a given target sequence, thereby minimizing promiscuous hybridization. Hybridization temperature varies inversely with nucleic acid member annealing efficiency, as does the concentration of organic solvents, e.g., formamide, that might be included in a hybridization mixture, while increases in salt concentration facilitate binding. Under stringent annealing conditions, longer nucleic acids, hybridize more efficiently than do shorter ones, which are sufficient under more permissive conditions.

As used herein, the term “significant match”, when referring to nucleic acid sequences, means that two nucleic acid sequences exhibit at least 65% identity, at least 70%, at least 75%, at least 80%, at least 85%, and preferably, at least 90% identity, using comparison methods well known in the art (i.e., Altschul, S. F. et al., 1997, Nucl. Acids Res., 25:3389-3402; Schäffer, A. A. et al., 1999, Bioinformatics 15:1000-1011). As used herein, “significant match” encompasses non-contiguous or scattered identical nucleotides so long as the sequences exhibit at least 65%, and preferably, at least 70%, at least 75%, at least 80%, at least 85%, and preferably, at least 90% identity, when maximally aligned using alignment methods routine in the art.

As used herein, a “therapeutic agent” or “agent” refers to a compound that inhibits the ability of B2M to inhibit chondrocyte proliferation and includes agents which decrease the expression of B2M or increase or decrease one or more of the products of the B2M related genes and thereby inhibits the ability of B2M to inhibit chondrocyte proliferation or decreases the pathogenesis of OA. The invention provides for a “therapeutic agent” that 1) prevents the onset or pathogenesis of osteoarthritis; 2) reduces, delays, or eliminates osteoarthritis symptoms such as pain, swelling, weakness and loss of functional ability in the afflicted joints; 3) reduces, delays, or eliminates cartilage degeneration, and/or enhances chondrocyte metabolic activity and cell division rates; and/or 4) restores one or more expression profiles of the B2M gene or a B2M related gene or B2M or the product of the B2M related genes in a patient to a profile more similar to that of a normal individual when administered to a patient.

As used herein, the term “steroidal compound” refers to any of numerous naturally occurring or synthetic fat-soluble organic compounds having as a basis 17 carbon atoms arranged in four rings and including the sterols and bile acids, adrenal and sex hormones, certain natural drugs such as digitalis compounds, and the precursors of certain vitamins.

As used herein, the term “non-steroidal compound” refers to any compounds that does not belong to the category of “steroidal compound” as defined herein above.

As used herein, the term “increase in chondrocyte proliferation” refers to the increase of in-vitro chondrocyte cell proliferation rate in a sample by a candidate modulator (e.g., a non-steroidal compound) by at least 10%, preferably, at least 20%, or 25% or 50% or more. When determining the increase of chondrocyte cell proliferation, the sample contacted with a candidate modulator is compared to a control sample that is not contacted with a candidate modulator. As used herein, the term “selectively inhibiting B2M activity” refers to the ability of a candidate modulator to specifically inhibit the function or the expression of B2M gene or gene product. By specifically inhibiting the function of the B2M gene or gene product is meant the reduction of the activity of B2M wherein the normal function of B2M is to inhibit chondrocyte proliferation. This can be measured by measuring the chondrocyte proliferation in vitro in the presence of B2M alone as compared with the chondrocyte proliferation in vitro in the presence of B2M along with a candidate modulator. By specifically inhibiting expression of B2M is meant the decrease in expression of the B2M gene. Inhibition of B2M expression by a candidate modulator is measured against the inhibition of a control gene or gene product (e.g., albumin) by the same candidate modulator. A candidate modulator is said to “selectively inhibit B2M activity” if the inhibition of B2M activity (either via an increase in chondrocyte proliferation or the inhibition of B2M gene expression) is at least 10% more, or at least 25% more, or at least 50% more, or at least 100% more than the inhibition of the relevant control.

As used herein, the term “up regulated” or “up regulation” refers to differential expression wherein the RNA, including mRNA in a first sample is expressed in greater amounts as compared with a second sample and includes increased differential expression of 1.5 fold, 2 fold, 2.5 fold, 3 fold etc.

As used herein, “inhibits” chondrocyte proliferation refers to the reduction in chondrocyte proliferation in the presence of B2M as compared to chondrocyte proliferation in the absence of B2M by at least 1%, 5%, 10% or more, for example, 20%, 30%, 40%, or 50%, 60%, 70%, 80%, 90% or more, as measured by the assay disclosed in Example 4.

Inhibitors

Variants

Variants of B2M or the products of the B2M related genes include insertions, deletions, conserved amino acid substitutions and non-conserved amino acid substitutions wherein the variant is capable of modulating the role of B2M in chondrocyte proliferation and/or the pathogenesis of OA.

One or more amino acid insertions or deletions may be introduced into peptide fragments of the invention. Amino acid insertions may consist of a single amino acid residue or sequential amino acid insertions ranging from 1-100, more particularly 1-50, more particularly 1-10, more particularly 1-5 amino acids in length. For example, amino acid insertions may be used so as to maintain the secondary or tertiary structure of B2M or the products of the B2M related genes and thus maintain ability of these proteins to bind to their target receptors, or interact with their wild type target proteins, while preventing or modulating the activity of these proteins in chondrocyte proliferation and/or the pathogenesis of OA.

Deletions may consist of single amino acid deletions or sequential amino acid deletions ranging from approximately 1-50 amino acids, preferably 1-10 amino acids, more preferably 1-5 amino acids and most preferably less than 5 amino acids.

The invention also contemplates isoforms of the peptide fragments of the invention. An isoform contains the same number and kinds of amino acids as a protein of the invention, but the isoform has a different molecular structure. The isoforms contemplated by the present invention include cyclic peptides. Isoforms may have the ability to bind to the specific receptor and/or preferentially or competitively bind to the specific receptor as compared to the wild type B2M or wildtype products of the B2M related genes.

The peptide variants of the invention also include homologs of the amino acid sequences of the invention and/or truncations thereof as described herein. Such homologs include peptides with an amino acid sequence having at least 70% preferably 75% more preferably 80%, most preferably 90% identity with the peptide fragments of the invention.

Mimetics

The mimetics of the invention should ideally be able to bind preferentially to the target of B2M or the target of the product(s) of the B2M related genes but should demonstrate a lesser ability to activate the pathogenesis of OA or inhibit chondrocyte proliferation. By this it is meant that the mimetic should ideally bind to a specific receptor with similar or greater affinity as compared with B2M or the wild type products of the B2M related genes, but prevents activation of said specific receptor, or demonstrate a lesser ability to activate OA pathogenesis or inhibit chondrocyte proliferation. The mimetic also should not, to any significant degree, bind to molecules that the wild type B2M or the products of the B2M related genes do not bind to. Of course, by careful screening, mimetics according to the invention may be chosen to possess selected properties of the wild type proteins, to suit the application of choice.

In order to be useful in providing potential lead drug compounds and/or useful in vitro tools for further chondrocyte studies, mimetics of the invention should bind to the target molecule with an affinity of at least 1 mM, preferably, 1 μM, more preferably at least 50 nM, more preferably at least 1 μM, most preferably 100 nM or less.

Mimetics may also contain amino acids other than the 20 nucleotide-encoded amino acids, wherein said amino acids are modified either by natural processes, such as by post-translational processing, or by chemical modification or chemicals synthesis techniques which are well known in the art. The inclusion of such amino acids may resolve a problem that is inherent in the pharmaceutical use of the wild type proteins, which are generally degraded and/or eliminated rapidly in vivo.

Examples of known modifications which may commonly be present in peptides of the present invention are glycosylation, glycation, hydroxylation, lipid attachment, sulphation, gamma-carboxylation of glutamic acid residues, and ADP-ribosylation, for instance. Other potential modifications include acetylation, acylation, ADP-ribosylation, amidation, covalent attachment of flavin, covalent attachment of a haeme moiety, covalent attachment of a nucleotide or nucleotide derivative, covalent attachment of a lipid or lipid derivative, covalent attachment of phosphotidylinositol, cross-linking, cyclization, disulphide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulphation, transfer-RNA mediated addition of amino acids to proteins such as arginylation and ubiquitination.

Modifications can occur anywhere in the protein, including in the peptide backbone, the amino acid side-chains and the amino or carboxyl termini. In fact, blockage of the amino or carboxyl group in a peptide, or both, by a covalent modification, is common in naturally-occurring synthetic peptides and such modifications may also be present in peptides of the present invention.

Antibodies

Antibodies to B2M or one or more functional epitopes of B2M or one or more of the products of the B2M related genes or one or more of the functional epitopes described herein may be readily prepared by one skilled in the art given the disclosure provided herein and can be used for assaying purposes, therapeutic purposes or for diagnostic purposes. Antibodies to specific receptor molecules found to interact with the peptide fragments are also encompassed within the present invention and can be used for therapeutic purposes.

When a protein or fragment of a protein is used to immunize a host animal, numerous regions of the protein may induce the production of antibodies which bind specifically to a given region or three-dimensional structure on the protein; these regions or structures are referred to as epitopes or antigenic determinants. As used herein, “antigenic fragments” refers portions of a polypeptide that contains one or more epitopes. Epitopes can be linear, comprising essentially a linear sequence from the antigen, or conformational, comprising sequences which are genetically separated by other sequences but come together structurally at the binding site for the polypeptide ligand. “Antigenic fragments” may be 5000, 1000, 500, 400, 300, 200, 100, 50 or 25 or 20 or 10 or 5 amino acids in length.

B2M or a product of the B2M related gene or antigenic portion thereof can be used to prepare specific antibodies. Conventional methods can be used to prepare the antibodies. For example, by using a protein of the invention, polyclonal antisera or monoclonal antibodies can be made using standard methods. This invention also contemplates chimeric antibody molecules, known to those skilled in the art.

Antisense RNA,

One aspect of the invention, a n inhibitor can be a sequence complementary to B2M or a B2M related cDNA or activator of B2M activity in antisense therapy. As used herein, antisense therapy refers to administration or in situ generation of oligonucleotide molecules or their derivatives which specifically hybridize (e.g., bind) under cellular conditions with the cellular mRNA and/or genomic DNA encoding B2M or a B2M related gene or activator of B2M activity, thereby inhibiting transcription and/or translation of the gene. The binding may be by conventional base pair complementarity, or, for example, in the case of binding to DNA duplexes, through specific interactions in the major groove of the double helix. In general, antisense therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the cellular mRNA. Alternatively, the antisense construct is an oligonucleotide probe which is generated ex vivo and which, when introduced into the cell, causes inhibition of expression by hybridizing with the mRNA and/or genomic sequences of a subject nucleic acid. Such oligonucleotide probes are preferably modified oligonucleotides which are resistant to endogenous nucleases, e.g., exonucleases and/or endonucleases, and are therefore stable in vivo and in vitro. Exemplary nucleic acid molecules for use as antisense oligonucleotides are phosphoramidate, phosphorothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668. With respect to antisense DNA, oligodeoxyribonucleotides derived from the translation initiation site, e.g., between the −10 and +10 regions of the nucleotide sequence of interest, are preferred.

Antisense approaches involve the design of oligonucleotides (either DNA or RNA) that are complementary to B2M or a B2M-related mRNA. The antisense oligonucleotides will bind to B2M or to B2M-related mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the B2M or a B2M related mRNA, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. (Wagner, R. 1994. Nature 372:333). Therefore, oligonucleotides complementary to either the 5′ or 3′ untranslated, non-coding regions of the B2M or B2M-related gene could be used in an antisense approach to inhibit translation of endogenous mRNA. Oligonucleotides complementary to the 5′ untranslated region of the mRNA should include the complement of the AUG start codon. Antisense oligonucleotides complementary to mRNA coding regions are typically less efficient inhibitors of translation but could also be used in accordance with the invention. Whether designed to hybridize to the 5′, 3′, or coding region of subject mRNA, antisense nucleic acids should be at least six nucleotides in length, and are preferably less that about 100 and more preferably less than about 50, 25, 17 or 10 nucleotides in length.

Regardless of the choice of target sequence, it is preferred that in vitro studies are first performed to quantitate the ability of the antisense oligonucleotide to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO 88/098 10, published Dec. 15, 1988) or the blood-brain barrier (see, e.g., PCT Publication No. WO 89/10 134, published Apr. 25, 1988), hybridization-triggered cleavage agents (See, e.g., Krol et al., 1988, BioTechniques 6:958-976), or intercalating agents (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytriethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Peny-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methyiphosphonate, an alkyl phosphotriester, and a formacetal or analog thereof.

In yet a further embodiment, the antisense oligonucleotide is an a-anomeric oligonucleotide. An a-anomeric oligonucleotide forms specific double-stranded hybrids with complementary RNA in which, contrary to the usual n-units, the strands run parallel to each other (Gautier et al, 1987, Nucl. Acids Res. 15:6625-6641). The oligonucleotide is a 2′-O-methylribonucleotide (Inoue et al., 1987, Nucl. Acids Res. 15:6131-12148), or a chimeric RNA-DNA analogue (Jnoue et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate olgonucleotides can be prepared by use of controlled pore glass polymer supports (Sarin et al., 1988, Proc. Natl. Acad. Sci. U.S.A. 85:7448-7451), etc.

While antisense nucleotides complementary to a coding region sequence can be used, those complementary to the transcribed untranslated region and to the region comprising the initiating methionine are most preferred.

The antisense molecules can be delivered to cells which express the target nucleic acid in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation on endogenous mRNAs. Therefore, a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous transcripts and thereby prevent translation of the target mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-3 10), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et at, 1982, Nature 296:39-42), etc. Any type of plasmid, cosmid, YAC or viral vector can be used to prepare the recombinant DNA construct which can be introduced directly into the tissue site; e.g., the choroid plexus or hypothalamus. Alternatively, viral vectors can be used which selectively infect the desired tissue (e.g., for brain, herpesvirus vectors may be used), in which case administration may be accomplished by another route (e.g., systemically).

In another aspect of the invention, ribozyme molecules designed to catalytically cleave B2M or B2M-related mRNA transcripts can be used to prevent translation and expression of B2M or B2M-related proteins (See, e.g., PCT International Publication WO90/11364, published Oct. 4, 1990; Sarver et al., 1990, Science 247:1222-1225 and U.S. Pat. No. 5,093,246). While ribozymes that cleave mRNA at site specific recognition sequences can be used to destroy B2M or B2M-related mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591. Preferably the ribozyme is engineered so that the cleavage recognition site is located near the 5′ end of the target mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. W088/04300 by University Patents Inc.; Been and Cech, 1986, Cell, 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in a target gene.

As in the antisense approach, the ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous messages and inhibit translation. Because ribozymes, unlike antisense molecules, are catalytic, a lower intracellular concentration is required for efficiency.

In yet another embodiment, the present invention also relates to a method of mediating RNA interference of B2M or B2M-related mRNA in a cell or organism (e.g., mammal such as a mouse or a human). In one embodiment, double stranded RNA (RNAi) of about 21 to about 23 nt which targets the mRNA to be degraded is introduced into the cell or organism. The cell or organism is maintained under conditions under which degradation of the mRNA occurs, thereby mediating RNA interference of the B2M or B2M-related mRNA in the cell or organism. The production of RNAi molecules in vivo and in vitro and their methods of use are described in U.S. Pat. No. 6,506,559, WO 01/36646, WO 00/44895, US2002/0162126, US2002/0086356, US 2003/0108923, WO 02/44321, WO 02/055693, WO 02/055692 and WO 03/006477.

Antisense RNA, DNA, ribozyme and RNAi molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ 0-methyl rather than phosphodiesterase linkages within the oligodeoxyribonucleotide backbone.

Therapeutic Agents

A useful therapeutic agent according to the invention is an inhibitor which can inhibit the role of B2M in the pathogenesis of OA and/or inhibit the ability of B2M to inhibit proliferation of chondrocytes. Preferably, a therapeutic agent can inhibit B2M activity on chondrocytes by greater than 1.0-fold, more preferably, 1.5-5-fold, and most preferably, 5-100-fold, as compared to an untreated chondrocytes.

In another embodiment, a therapeutic agent according to the invention can ameliorate at least one of the symptoms and/or changes associated with osteoarthritis including cartilage degeneration, or pain, swelling, weakness and/or loss of functional ability in the afflicted joints, associated with cartilage degeneration.

The candidate therapeutic agent may be a synthetic compound, or a mixture of compounds, or may be a natural product (e.g. a plant extract or culture supernatant).

Candidate therapeutic agents or compounds from large libraries of synthetic or natural compounds can be screened. Numerous means are currently used for random and directed synthesis of saccharide, peptide, and nucleic acid-based compounds. Synthetic compound libraries are commercially available from a number of companies including Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Combinatorial libraries are available and are prepared. Alternatively, libraries of natural compounds in the form of bacterial, fungal, plant and animal extracts are available from e.g., Pan Laboratories (Bothell, Wash.) or MycoSearch (NC), or are readily produceable by methods well known in the art. Additionally, natural and synthetically produced libraries and compounds are readily modified through conventional chemical, physical, and biochemical means.

Useful compounds may be found within numerous chemical classes. Useful compounds may be organic compounds, or small organic compounds. Small organic compounds have a molecular weight of more than 50 yet less than about 2,500 daltons, preferably less than about 750, more preferably less than about 350 daltons. Exemplary classes include heterocycles, peptides, saccharides, steroids, and the like. The compounds may be modified to enhance efficacy, stability, pharmaceutical compatibility, and the like. Structural identification of an agent may be used to identify, generate, or screen additional agents. For example, where peptide agents are identified, they may be modified in a variety of ways to enhance their stability, such as using an unnatural amino acid, such as a D-amino acid, particularly D-alanine, by functionalizing the amino or carboxylic terminus, e.g. for the amino group, acylation or alkylation, and for the carboxyl group, esterification or amidification, or the like.

Dosage and Administration

Therapeutic agents of the invention are administered to a patient, preferably in a biologically compatible solution or a pharmaceutically acceptable delivery vehicle, by ingestion, injection, inhalation or any number of other methods routine in the art. The dosages administered will vary from patient to patient. A “therapeutically effective dose” is determined, for example, by the level of enhancement of function (e.g., increased or decreased chondrocyte proliferation and or increase or decrease OA pathogenesis.

A therapeutic agent according to the invention is administered in a single dose. This dosage may be repeated daily, weekly, monthly, yearly, or as considered appropriate by the treating physician.

Pharmaceutical Compositions

The invention provides for compositions comprising a therapeutic agent according to the invention admixed with a physiologically compatible carrier. As used herein, “physiologically compatible carrier” refers to a physiologically acceptable diluent such as water, phosphate buffered saline, or saline, and further may include an adjuvant. Adjuvants such as incomplete Freund's adjuvant, aluminum phosphate, aluminum hydroxide, or alum are materials well known in the art.

The invention also provides for pharmaceutical compositions. In addition to the active ingredients, these pharmaceutical compositions may contain suitable pharmaceutically acceptable carrier preparations which is used pharmaceutically.

Pharmaceutical compositions for oral administration are formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use are obtained through a combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl cellulose, hydroxypropylmethyl-cellulose, or sodium carboxymethyl cellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Drug cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which are used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the invention may be formulated in aqueous solutions, preferably in physiologically compatible buffers such as Hank's solution, Ringer' solution, or physiologically buffered saline. Aqueous injection suspensions may contain substances which increase the viscosity of the suspension, such as sodium carboxymethyl cellulose, sorbitol, or dextran. Additionally, suspensions of the active solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For nasal administration, penetrants appropriate to the particular barrier to be permeated are used in the formulation. Such penetrants are generally known in the art.

The pharmaceutical compositions of the present invention may be manufactured in a manner known in the art, e.g. by means of conventional mixing, dissolving, granulating, dragee-making, levitating, emulsifying, encapsulating, entrapping or lyophilizing processes.

The pharmaceutical composition may be provided as a salt and are formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5 that is combined with buffer prior to use.

After pharmaceutical compositions comprising a therapeutic agent of the invention formulated in a acceptable carrier have been prepared, they are placed in an appropriate container and labeled for treatment of an indicated condition with information including amount, frequency and method of administration.

Identification of Inhibitors of B2M Activity

The invention teaches methods for the identification of inhibitors of B2M or B2M related activity. In one embodiment, inhibitors of B2M or B2M related activity are identified by their ability to increase chondrocyte proliferation in the presence of B2M, as disclosed in Example 4. In another embodiment the invention provides for the differential expression of genes identified below as biomarkers of B2M activity. In another embodiment the chondrocyte proliferation assay and the differential expression of the genes disclosed below permit the identification of inhibitors of B2M or B2M related activity.

The invention provides a set of 20 genes identifiable in chondrocytes (see Table 2) whose level of expression is upregulated by a concentration of B2M (0.1-10 μg/ml) that inhibits chondrocyte proliferation. These genes, or the products of these genes, in combination are therefore useful as biomarkers of B2M activity.

The invention also provides a set of 11 genes identifiable in chondrocytes (see Table 3) whose level of expression is down-regulated by a concentration of B2M (0.1-10 μg/ml) that inhibits chondrocyte proliferation. These genes, or the products of these genes, in combination are also useful as biomarkers of B2M activity.

It would be understood by a person skilled in the art that one or more, two or more, three or more, four or more, five or more etc of the 20 genes, or the products of these genes that are upregulated by B2M in combination are useful as biomarkers of B2M activity (see Table 2).

It would also be understood by a person skilled in the art that one or more, two or more, three or more, four or more, five or more etc of the 11 genes, or the products of these genes that are down-regulated by B2M in combination are useful as biomarkers of B2M activity (see Table 3).

It would also be understood by a person skilled in the art that one or more, two or more, three or more, four or more, five or more etc of the 20 genes that are upregulated by B2M or the products of these genes (see Table 2) and the one or more, two or more, three or more, four or more, five or more etc of the 11 genes or the products of these genes that are down-regulated by B2M are useful in combination as biomarkers of B2M activity.

More specifically the number of useful combinations is described (Feller, W. F., Intro to Probability Theory, 3^{rd} Ed. Volume 1, 1968, ed. J. Wiley) and can be calculated using the general formula:

20!/(n)! (20−n)!

where n is the number of genes to be selected for the combination and 20 is the number of genes upregulated by B2M to be considered.

For example there are

$$\frac{20 !}{2 ! ( 20 - 2 ) !} = \frac{\begin{matrix}20 \times 19 \times 18 \times 17 \times 16 \times 15 \; 14 \times 13 \times \\ 12 \times 11 \times 10 \times 9 \times 8 \times 7 \times 6 \times 5 \times 4 \times 3 \times 2 \times 1\end{matrix}}{\begin{matrix}( 2 \times 1 ) ( 18 \times 17 \times 16 \times 15 \; 14 \times 13 \times 12 \times \\ 11 \times 10 \times 9 \times 8 \times 7 \times 6 \times 5 \times 4 \times 3 \times 2 \times 1 )\end{matrix}}$$

possible combinations of two B2M upregulated genes wherein the combination of the two B2M upregulated genes are useful as biomarkers of B2M activity.

Efficacy of Osteoarthritis Therapy Using Defined Therapeutic Agents

The efficacy of the therapy using any of the therapeutic agents according to the invention is determined by a medical practitioner. This determination may be related to alleviating osteoarthritis symptoms such as pain, swelling, weakness and loss of functional ability in the afflicted joint(s), and/or criteria for osteoarthritis diagnosis and staging described in Marshall (1996, supra).

Methods

Human synovial fluid and cartilage samples. Human OA synovial fluid was aspirated and cartilage samples were obtained from knee joints at arthroscopy or total knee replacement. OA severity was graded according to the system described by Marshall. (Marshall K W. The case for a simple method of grading osteoarthritis severity at arthroscopy. J Rheumatol, 1996:23(4) 582-85).

Briefly, each of the six knee articular surfaces was assigned a cartilage grade with points based on the worst lesion seen on each particular surface. Grade 0 is normal (0 points), Grade I cartilage is soft or swollen but the articular surface is intact (1 point). In Grade II lesions, the cartilage surface is not intact but the lesion does not extend down to subchondral bone (2 points). Grade III damage extends to subchondral bone but the bone is neither eroded nor eburnated (3 points). In Grade IV lesions, there is eburnation of or erosion into bone (4 points). A global OA score is calculated by summing the points from all six cartilage surfaces. Based on the total score, each patient is then categorized into one of four OA groups: mild (1-6), moderate (7-12), marked (13-18), and severe (>18). Normal synovial fluid samples were collected from volunteers with no history of knee injury or arthritis. All synovial fluids were taken as undiluted samples.

Human fetal femoral cartilage samples were collected from a pool of aborted fetuses (8-12 wk) and stored immediately in liquid nitrogen. Human mild and severe OA cartilage samples (graded as described above) were collected at the time of arthroscopy or total knee replacement and stored in liquid nitrogen immediately for subsequent RNA isolation and RT-PCR analysis. For tissue culture, severe OA cartilage samples were collected at the time of total knee replacement and used immediately.

RT-PCR.

Pooled fetal, mild (six patients) or severe (three patients) OA cartilage samples were subjected to total RNA extraction using TRIzol (GIBCO) reagent. (Marshall, K. W., Zhang, H., Hwang, D. M., Lee, M., and Liew, C. C. Profiling genes expressed in human fetal cartilage by expressed sequence tags (ESTs). Osteoarthritis Cartilage 2001 (submitted)).

Reverse transcription was carried out using 1 μg total RNA with oligo-dT and Superscript II reverse transcriptase (GIBCO). B2M specific primers were designed at positions 167 and 581 in the B2M nucleotide sequence (NM_{—}004048). The forward primer is between 167-186 (5′-CCA TCC GAC ATT GAA GTT GA-3′ (SEQ ID NO: 1)) and the reverse primer is between 561-581 (5′-TGG AGC AAC CTG CTC AGA TA-3′ (SEQ ID NO: 2)). This results in a PCR product that is 415 bp in size. PCR reaction was carried out at an annealing temperature of 54° C. for 30 cycles. Glyceraldehyde-3-Phosphate Dehydrogenase (GAPDH, NM_{—}002046) was used as an internal control for mRNA amplification. The forward primer is 5′-TGG TAT CGT GGA AGG ACT CAT-3′ (SEQ ID NO: 3), and the reverse primer is 5′-GTG GGT GTC GCT GTT GAA GTC-3′ (SEQ ID NO: 4), giving a 370 bp size PCR product.

Cartilage Organ Culture.

Human severe OA cartilage slices (˜10×12 mm2 surface area/slice) were cultured at one slice/well in a 24-well plate in DMEM (Dulbecco's modified Eagle medium, GIBCO), with 10% FCS (fetal calf serum, GIBCO), 100 units/ml penicillin and 100 mg/ml streptomycin (GIBCO) (DMEM++) at 37° C. in a humidified atmosphere of 5% CO2. (Doherty P J, Zhang H, Trembley L, Manolopoulos V and Marshall K W. Resurfacing of articular cartilage explants with genetically-modified human chondrocytes in vitro. Osteoarthritis and Cartilage 1998;6:153-160).

Cultured medium (20 μl) was then collected at different time points for B2M ELISA.

ELISA.

B2M levels in synovial fluid and cartilage organ cultured medium were measured using a beta-2 microglobulin enzyme immunoassay test kit (ALPCO) according to the provided protocol. Briefly, 10 μl of sample or standard controls (provided) were mixed with 1.0 ml sample diluent. Subsequently, 5 μl of the diluted samples and diluted controls were placed into microtiter wells (96-well coated with mouse monoclonal anti-human B2M antibody). After applying the samples, 200 μl of sample diluent was added into each well and thoroughly mixed for 30 sec. at 200 rpm on a plate rotator. The samples were then incubated at 37° C. for 30 min. The incubation mixture was then removed and the wells were rinsed with running distilled water five times. 200 μl of Enzyme Conjugate Reagent (sheep anti-human B2M horseradish peroxidase conjugate) was then added into each well, mixed gently for 10 sec., and incubated at 37° C. for 30 min. The contents were then removed and the wells were washed. 200 μl of freshly mixed H2O2/TMB solution was then added into each well, mixed for 10 sec., and incubated at room temperature in the dark for 20 min. The reaction was stopped by adding 50 μl stop solution (2N HCl) to each well, mixed for 10 sec. and care taken to make sure that all blue colour changed to yellow colour. Absorbance was read at 450 nm with a microplate reader (BIO-TEK Instruments). All measurements were done in duplicate.

A standard curve was plotted for each experiment. The sample B2M concentration was determined by using the mean absorbance of the sample to find the corresponding concentration of B2M in μg/ml on the standard curve.

Cell Culture.

Chondrocytes cultured for proliferation assay Chondrocytes were retrieved from human severe OA cartilage slices as previously described. (Doherty P J, Zhang H, Trembley L, Manolopoulos V and Marshall K W. Resurfacing of articular cartilage explants with genetically-modified human chondrocytes in vitro. Osteoarthritis and Cartilage 1998;6:153-160).

Cells were then washed, counted and seeded at 1×104 cells/well in a flat-bottomed 96-well plate (Coming) in DMEM++. After cells attached to the plate, they were washed with DMEM only, and then incubated in DMEM with or without 10% FCS along with different concentrations (0-10 μg/ml) of B2M (Sigma) for 48 hr. Cell number in each well was then determined by adding 10 μl of WST-1 (a tetrazolium salt that can be cleaved to formazan by mitochondrial dehydrogenases in live cells, Roche) to each well, mixed thoroughly for 1 min. and incubated at 37° C. for 1.5 hr. Then the plate was scanned by a microplate autoreader (BIO-TEK Instruments) at an absorbance of 450 nm. The number of viable cells is reflected by the amount of formazan formed which is quantified by measuring absorbance at 450 nm . (Lang I, Hoffman C, Olip H, Pabst M A, Hahn T, Dohr G, Desoye G. Differential mitogenic responses of human macrovascular and microvascular endothelial cells to cytokines underline their phenotypic heterogeneity. Cell Prolif 2001;34:143-55).

Experiments were performed in triplicate and repeated with three different severe OA cartilage samples.

Chondrocytes Cultured for Microarray Assay

Chondrocytes derived from severe OA cartilage were seeded at 6.5×104/well (3.2×104/ml) in a 6-well plate (Becton Dickinson). After cells attached to the plate, they were washed with DMEM and cultured with DMEM (no FCS), with or without 10 μg/ml B2M for 72 hr. After incubation, cells were washed twice with GBSS (Gey's Balanced Salt Solution, GIBCO) and then subjected for total RNA isolation with TRIzol reagent. RNA concentration and quality were checked by measuring OD260/OD280 nm, and gel electrophoresis.

Microarray Assay.

In-house microarray slide preparation PCR products (˜40 μl) of 5184 cDNA clones derived from cDNA libraries of mild and severe OA cartilage were precipitated, washed and resuspended in 20 μl 3×SSC. Samples were then spotted in duplicate onto aminosilane coated slides (25×75 mm, Corning) using a robotic GMS 417 arrayer (Genetic MicroSystems, MA). The size of the array area was 18×36 mm. After spotting, slides were rehydrated, heating blocked and UV crosslinked (Stratagene, Stratalinker, 65 mJ). To prevent non-specific probe binding, slides were treated as described by DeRisi et al. (Schena M, Shalon D, Davis R W, Brown P O. Quantitative monitoring of gene expression patterns with a complementary DNA microarray. Science 1995;270: 467-70). (DeRisi J, Renland L, Brown P O, Bittner M L, Meltzer P S, Ray M, et al. Use of a cDNA microarray to analyse gene expression patterns in human cancer. Nature Genetics 1996;14:457-60).

Probe Preparation

Probes used for microarray hybridization were prepared using the mRNA amplification method established by Wang et al (Wang E, Miller L D, Ohnmacht G A, Liu E T, Marincola F M. High-fidelity mRNA amplification for gene profiling Nature Biotechnology 2000;18:457-9). with a few modifications. Briefly, 1 μg total RNA was used for first strand cDNA synthesis with oligo-dT (15)-T7 primer (5′-AAA CGA CGG CCA GTG AAT TGT AAT ACG ACT CAC TAT AGG CGC T(15)-3′ (SEQ ID NO: 5)) and template switch oligo primer (5′-AAG CAG TGG TAT CAA CGC AGA GTA CGC GGG-3′ (SEQ ID NO: 6)). Second strand synthesis was carried out with Advantage Polymerase Kit (Clontech). Double strand cDNA was cleaned with Bio-6 Chromatograph column (Bio-Rad). In vitro transcription for aRNA was done with T7 Megascript Kit (Ambion) and purified RNA with TRIzol reagent. One microgram aRNA was used for second round amplification. Three micrograms of amplified aRNA was used for probe labeling by reverse transcription with 1 mM Cy3 or Cy5 (Pharmacia).

Hybridization

The protocol used for hybridization was based on that provided by Hegde et al. (Hegde P, Qi R, Abernathy K, Gay C, Dharap S, Gaspard R, et al. The institute for Genomic Research, Rockville, Md. Protocol is available at the web address www.tigr.org/tdb/microarray/conciseguide) Briefly, each slide was prehybridized with prehybridization buffer (5×SSC, 0.1% SDS and 1% bovine serum albumin-Sigma) for 45 min at 42° C. The hybridization solution was prepared as follows: Cy3 and Cy5 labeled probes were pooled together, 50 μg COT1-DNA (Life Technologies), 1 μgPoly (A)-DNA (40-60) (Pharmacia) and 18 μl 2× hybridization buffer (50% formamide, 10×SSC, and 0.2% SDS) were added. This hybridization solution was then applied onto each prehybridized microarray slide, avoiding air bubbles, and a cover-slip (20×40 mm Corning) was placed on each slide. The slides were then placed in a sealed hybridization chamber (International Telechem) and 40 μl H2O was added to the chamber. The chamber was incubated in a 42° C. water bath for 16-20 hrs. After hybridization, the slides were carefully removed from the chamber and placed into a low-stringency washing buffer (1×SSC, 0.2% SDS) at 42° C. for 4 min. The cover-slips were gently removed from the slides. The slides were then washed in a high-stringency buffer (0.1×SSC, 0.2% SDS) at RT for 4 min. Finally, the slides were washed in 0.1×SSC for 4 min and allowed to air dry.

Data Collection and Analysis

Each slide was scanned using a GMS 418 Array Scanner (Genetic MicroSystems, MA), first in the Cy5 channel and then the Cy3 channel. Data from each fluorescence channel was collected and stored as a separate 16-bit TIFF image. The images were then analyzed using the ScanAlyze program. (Eisen M. ScanAlyze User Manual version 2.32. 1999 Stanford University). The incorporated image (Cy5 and Cy3) then underwent a gridding process, which allows for manual adjustment of each grid for each spot. The fluorescence intensity and background-subtracted hybridization intensity of each spot were collected. The ratio of measured mean pixel intensities of Cy5 to Cy3 was calculated. To identify differentially expressed genes, the data generated must be normalized to adjust for differences in labeling and detection efficiencies for the fluorescent labels. A linear regression approach was used for normalization. This approach assumes that for closely related samples, one would expect many of the genes to be expressed at nearly constant levels. Thus, a scatter plot of the measured Cy5 versus Cy3 intensities should have a slope of one. The average of the ratios (Cy5/Cy3) of spots was calculated and used to rescale the data and adjust the slope to one. A post-normalization cutoff of two-fold up- or down-regulation was used to define differentially expressed genes. (Hegde P, Qi R, Abernathy K, Gay C, Dharap S, Gaspard R, et al. The institute for Genomic Research, Rockville, Md. Protocol is available at the web address www.tigr.org/tdb/microarray/conciseguide (Chen Y, Dougherty E R, and Bittner M L. Ratio-based decisions and the quantitative analysis of cDNA microarray images. J. Biomed. Optics 1997;24:364-74).

Statistical Analysis.

Statistical significance was assessed by Student's t-test. A p value less than 0.05 was considered significant.

### EXAMPLES

### Example 1

### Expression of B2M mRNA in Human Fetal, Mild and Severe OA Cartilage

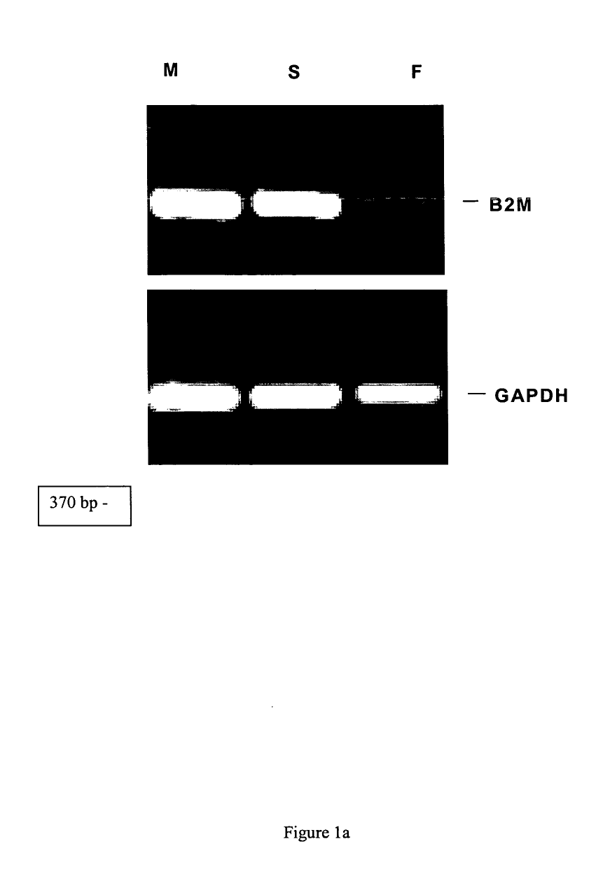

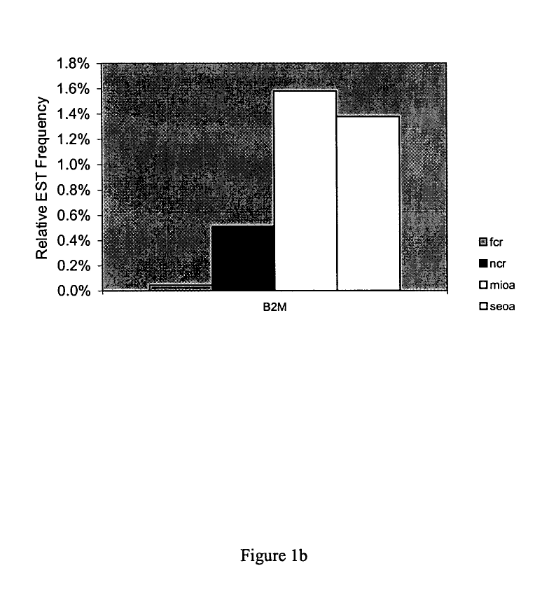

Fetal, mild and severe OA cartilage samples were tested for their B2M mRNA expression. The level of B2M mRNA was determined by RT-PCR. FIG. 1a shows that mild and severe OA cartilage express similar levels of B2M, and they are both higher than that found for fetal cartilage. This result is consistent with our previous EST findings that B2M expression was significantly higher in mild (1.58%) and severe OA (1.38%) than in fetal (0.04%) and normal adult cartilage (0.50%), as shown in FIG. 1b. Normal cartilage samples were provided by the donor program at the Department of Orthopaedics and Rehabilitation, University of Miami. The samples were harvested within 12 hr, from the femoral condyle of patients who had died of electrocution and blunt injuries.)

### Example 2

### B2M Levels in Normal and OA Synovial Fluid

Elevated B2M levels in SF have been observed in most rheumatoid arthritis patients, however, the relative level of B2M in osteoarthritic SF is not yet clear. (Moe S M, Singh G K and Bailey A M. beta2-microglobulin induces MMP-1 but not TIMP-1 expression in human synovial fibroblasts. Kidney International 2000;57:2023-34) (Williams R C Jr, Malone C C, Nissen M H, Aono F M, Vachula M, Van Epps D E. Des-Lys58-beta 2 m and native beta 2 m in rheumatoid arthritis serum amd synovial fluid. Clin Exp Rheumatol 1994;12:635-41). (Alenius G M, Stegmayr B G, Dahlqvist S R. Renal abnormalities in a population of patients with psoriatic arthritis. Scand J Rheumatol 2001;30:271-4).

A total of fifty-five SF samples were tested, the results of which are described in Table 1. The severity of OA was based on the total score of the joint at the time of arthroscopy or total knee replacement. (Marshall K W. The case for a simple method of grading osteoarthritis severity at arthroscopy. J Rheumatol, 1996:23(4) 582-85).

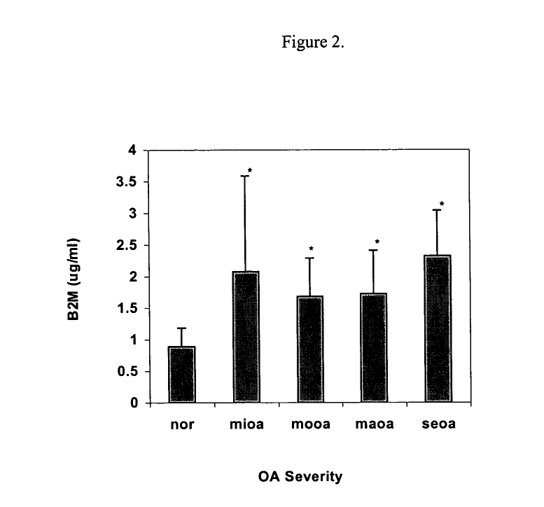

FIG. 2 illustrates the average B2M levels detected in normal, mild, moderate, marked and severe OA SF. B2M levels in the synovial fluid from differing severities of OA is significantly higher than in the normal group (p<0.05). However, there is no significant difference in B2M levels between the differing OA severities.

### Example 3

### Secretion of B2M by OA Cartilage in Vitro

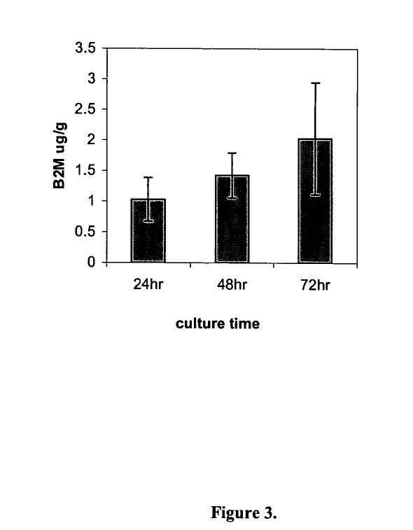

To investigate if OA cartilage secretes B2M, an in vitro cartilage organ culture was set up to detect B2M levels in cultured medium. Severe OA cartilage slices were cultured and medium was collected at different time points and tested for B2M. FIG. 3 shows that the release of B2M is detectable in 24 hr cultures and continues to increase during the 72 hr study period. Student t-test results show that the B2M amount at 72 hr is close to being statisticaly significant (p=0.051). At the 72 hr point, the B2M generated was 2.1 μg/g cartilage on average. The results were based on three experimental runs, each using cartilage from a different donor.

### Example 4

### B2M Effect on OA Chondrocyte Proliferation

B2M has been shown to have growth stimulating effects on osteoblasts, but not on synovial fibroblasts. (Migita K., Eguchi K, Tominaga M, Origuchi T, kawabe Y, and Nagataki S. β2-Microglobulin induces Stromelysin production by human synovial fibroblasts. Bioch. and Biophy. Res. Commu. 1997;239:621-5).

(Evans D B, Thavarajan M, kanis J A. Immunoreactivity and proliferative actions of β2-microglobulin on human bone-derived cells in vitro. Biochem Biophys Res Comm 1991;175:795-803).

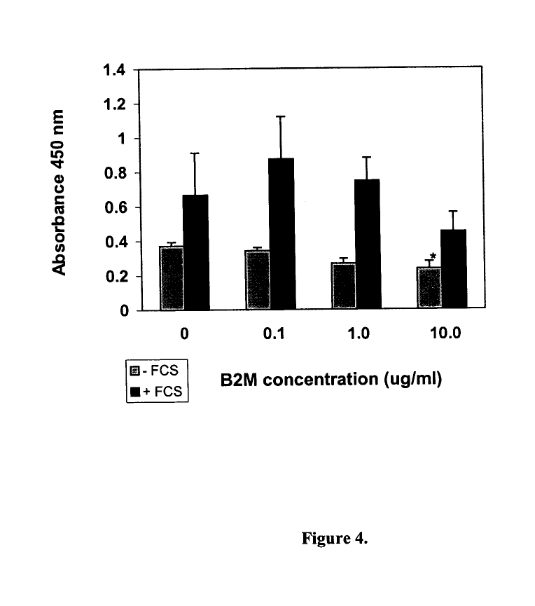

To investigate whether B2M has any effect on chondrocyte proliferation, primary cultured human severe OA chondrocytes were incubated with increasing concentrations of B2M in media with or without FCS for 48 hr. Cell numbers were then quantitatively estimated by their ability to cleave WST-1, which is reflected as the absorbance at 450 nm. As shown in FIG. 4, B2M did not stimulate chondrocyte proliferation. On the contrary, it showed an initial inhibitory effect at 1.0 μg/ml, with the effect reaching significance at 10.0 μg/ml (no FCS group). There was no significant difference observed among the B2M group treated with FCS, although there seemed to be a decrease in cell number at 10.0 μg/ml.

### Example 5

### B2M Effect on OA Chondrocyte Gene Expression

Previous studies on B2M amyloidosis have suggested that B2M is involved in cartilage destruction, as it induces stromelysin and MMP-1 in human synovial fibroblasts. (Migita K., Eguchi K, Tominaga M, Origuchi T, kawabe Y, and Nagataki S. β2-Microglobulin induces Stromelysin production by human synovial fibroblasts. Bioch. and Biophy. Res. Commu. 1997;239:621-5). (Migita K., Eguchi K, Tominaga M, Origuchi T, kawabe Y, and Nagataki S. β2-Microglobulin induces Stromelysin production by human synovial fibroblasts. Bioch. and Biophy. Res. Commu. 1997;239:621-5. Migita K., Tominaga M, Tominaga M, kawabe Y, Aoyagi T, Urayama S, Yamasaki S, Hida A, Kawakmi A, and Eguchi K. Induction of cyclooxygenase-2 in human synovial cells by β2-microglobulin. Kidney International 1999;55:572-8). (Moe S M, Singh G K and Bailey A M. beta2-microglobulin induces MMP-1 but not TIMP-1 expression in human synovial fibroblasts. Kidney International 2000;57:2023-34).

Here, we used microarray technology to detect chondrocyte genes regulated by B2M. Severe OA chondrocytes were cultured with and without 10 μg/ml B2M in serum free media for 72 hr. One microgram of total RNA isolated from the cells (−/+B2M) was used for mRNA amplification, fluoresence dye labeling and microarray hybridization. The DNA microarray contained duplicated 5184 cDNA elements, including 1842 known genes, 743 with no significant match and 2599 ESTs.

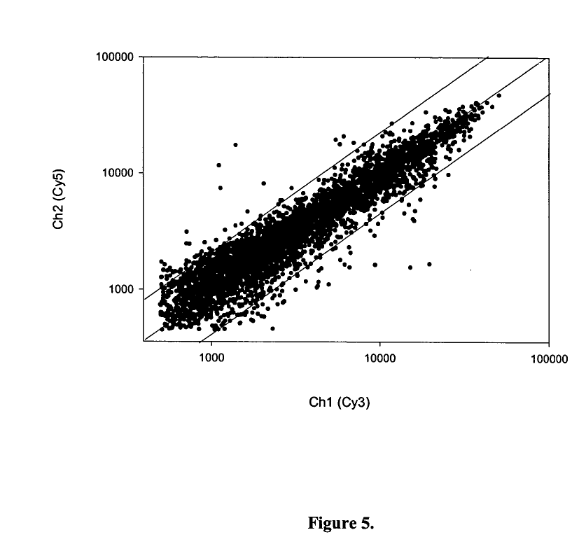

The scatter plot (FIG. 5) shows the relative fluorescence intensities in each of the two scanned channels. The ratio between channel 2 (cy5, red) and channel 1 (cy3, green) fluorescent intensities after normalization was used to define differentially expressed genes. As shown in FIG. 5, most spots have a ratio close to 1.0, suggesting that the expression of the genes they represent was not regulated by B2M.

Genes up-regulated by B2M have a ratio>2, while genes down-regulated by B2M have a ratio<0.5. Tables 2 and 3 list genes that were up and down-regulated at least two-fold by B2M (based on two experiments, with cross-labeling, where non-B2M treated were labeled with Cy5 and B2M treated were labled with Cy3). There were twenty genes found to be up-regulated by B2M. YKL-39, collagen type III, lumican, manganese superoxide dismutase and SP-100 are some of the known genes that were up-regulated by B2M. Eleven genes were found to be down-regulated by B2M.

### Example 6

### Identification of Variants of B2M Gene Activity

This example demonstrates the use of the methods disclosed herein to identify variants of B2M of the claimed invention. Human chondrocytes isolated from normal or severe OA patients are seeded at 1×10⁴ cells/well in triplicate into a 96 well plate. Cells are cultured without FCS but with 10 ug/ml, 20 ug/ml, and 30 ug/ml of wildtype B2M along with the putative variant in similar concentrations for 48 hours. Control samples are run concurrently wherein chondrocytes seeded at 1×10⁴ are cultured without FCS, but with 10 ug/ml, 20 ug/ml and 30 ug/ml of B2M alone. To measure chondrocyte proliferation 10 ul of WST-1 is added to each well and the plate scanned by a microplate reader at an absorbance of 450 nm. Readings of wells wherein chondrocytes are incubated with B2M along with the putative variant are compared with wells wherein chondrocytes are incubated with B2M alone. Variants capable of reducing B2Ms ability to decrease chondrocyte proliferation are identified.

### Example 7

### Identification of Mimetics of B2M Gene Activity

This example demonstrates the use of the methods disclosed herein to identify mimetics of B2M of the claimed invention. Human chondrocytes isolated from normal or severe OA patients are seeded at 1×10⁴ cells/well in triplicate into a 96 well plate. Cells are cultured without FCS but with 10 ug/ml, 20 ug/ml, and 30 ug/ml of wildtype B2M along with the putative mimetics in similar concentrations for 48 hours. Control samples are run concurrently wherein chondrocytes seeded at 1×10⁴ are cultured without FCS, but with 10 ug/ml, 20 ug/ml and 30 u g/ml of B2M alone. To measure chondrocyte proliferation 10 ul of WST-1 is added to each well and the plate scanned by a microplate reader at an absorbance of 450 nm. Readings of wells wherein chondrocytes are incubated with B2M along with the putative variant are compared with wells wherein chondrocytes are incubated with B2M alone. Mimetics capable of reducing B2Ms ability to decrease chondrocyte proliferation are identified.

### Example 8

### Identification of Antibodies of B2M

This example demonstrates the use of the methods disclosed herein to identify antibodies of B2M of the claimed invention. Human chondrocytes isolated from normal or severe OA patients are seeded at 1×10⁴ cells/well in triplicate into a 96 well plate. Cells are cultured without FCS but with 10 ug/ml, 20 ug/ml, and 30 ug/ml of wildtype B2M along with an antibody of B2M or a functional epitope of B2M in concentrations of 0.1 ug/ml, 1 ug/ml or 10 ug/ml or 20 ug/ml for 48 hours. Control samples are run concurrently wherein chondrocytes seeded at 1×10⁴ are cultured without FCS, but with 10 ug/ml, 20 ug/ml and 30 ug/ml of B2M alone. To measure chondrocyte proliferation 10 ul of WST-1 is added to each well and the plate scanned by a microplate reader at an absorbance of 450 nm. Readings of wells wherein chondrocytes are incubated with B2M along with the putative antibodies are compared with wells wherein chondrocytes are incubated with B2M alone. Antibodies capable of reducing B2Ms ability to decrease chondrocyte proliferation are identified.

### Example 9

### Identification of Inhibitors of B2M Activity

This example demonstrates the use of the methods disclosed herein to identify inhibitors of the claimed invention. Human chondrocytes isolated from normal or severe OA patients are seeded at 1×10⁴ cells/well in triplicate into a 96 well plate. Cells are cultured without FCS but with 10 ug/ml, 20 ug/ml, and 30 ug/ml of B2M along with the putative inhibitor in concentrations of 0.1 ug/ml, 1 ug/ml or 10 ug/ml for 48 hours. The inhibitor can be a variant or mimetic of B2M, antisense oligonucleotide, RNAi, ribozyme or antibody. Control samples are run concurrently wherein chondrocytes seeded at 1×10⁴ are cultured without FCS, but with 10 ug/ml, 20 ug/ml and 30 ug/ml of B2M. To measure chondrocyte proliferation 10 ul of WST-1 is added to each well and the plate scanned by a microplate reader at an absorbance of 450 nm. Readings of wells wherein chondrocytes are incubated with B2M along with the putative inhibitor are compared with wells wherein chondrocytes are incubated with B2M alone. Inhibitors capable of reducing B2Ms ability to decrease chondroctye proliferation are identified.

### Example 10

### Identification of Inhibitors of B2M Activity by Measuring the Expression of the Genes of Table 2 and Table 3

This example demonstrates the use of the methods disclosed herein to identify inhibitors of the claimed invention. Human chondrocytes isolated from normal or severe OA patients are seeded at 1×10⁴ cells/well in triplicate into a 96 well plate. Cells are cultured without FCS but with 10 ug/ml, 20 ug/ml, and 30 ug/ml of B2M along with the putative inhibitor in concentrations of 0.1 ug/ml, 1 ug/ml or 10 ug/ml for 48 hours. The inhibitor can be a variant or mimetic of B2M, an antisense oligonucleotide, RNAi, ribozyme or antibody. Control samples are run concurrently wherein chondrocytes seeded at 1×10⁴ are cultured without FCS, but with 10 ug/ml, 20 ug/ml and 30 ug/ml of B2M. To measure the effect of the putative inhibitor on B2M activity, total mRNA is isolated from each culture of chondrocytes using, for example, an acid guanidinium-phenol-chloroform extraction method and polyA+ mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987). In a preferred embodiment, total RNA is extracted using TRIzol® reagent (GIBCO/BRL, Invitrogen Life Technologies, Cat. No. 15596). Purity and integrity of RNA is assessed by absorbance at 260/280 nm and agarose gel electrophoresis followed by inspection under ultraviolet light. Fluorescently labeled probes corresponding to each chondrocyte culture are then generated, denatured and hybridized to a microarray chip containing probes for each of the genes/ESTs disclosed in Table 2 or Table 3. Differential expression of the genes of Table 2 and 3 in chondrocytes treated with B2M and putative inhibitor as compared to chondrocytes treated with B2M alone is then determined as described in Example 5.

Differentially decreased expression of the genes of Table 2 and/or differentially increased expression of the genes of Table 3 identifies the putative inhibitor as an inhibitor of B2M activity.

All patents, patent applications, and published references cited herein are hereby incorporated by reference in their entireties. While this invention has been particularly shown and described with references to preferred embodiments thereof, it will be understood by those skilled in the art that various changes in form and details may be made therein without departing from the scope of the invention encompassed by the appended claims.

TABLE 1
B2M levels in normal and osteoarthritic synovial fluid by ELISA.

| | | Number of | Patient age | B2M (μg/ml) |
|---|---|---|---|---|
| OA severity | OA Score | patients tested | range (mean) | range (X ± SD) |
| normal | 0 | 9 | 17-36 (25) | 0.69-1.27 |
| | | | | (0.89 ± 0.29) |
| mild | 1-6 | 11 | 24-66 (38) | 1.00-5.49 |
| | | | | (2.07 ± 1.50) |
| moderate | 7-12 | 10 | 31-68 (47) | 0.54-2.72 |
| | | | | (1.68 ± 0.60) |
| marked | 13-18 | 16 | 37-83 (64) | 0.73-2.95 |
| | | | | (1.72 ± 0.68) |
| severe | >18 | 9 | 48-86 (68) | 1.30-3.6 |
| | | | | (2.32 ± 0.72) |

X ± SD: mean and standard deviation

TABLE 2
Twenty genes up-regulated by B2M in OA chondrocytes. Alphabetical
list of genes showing at least two-fold differential expression
in B2M treated chondrocytes (two experiments).

| | | Gene Bank or | Protein |
|---|---|---|---|
| | Fold | UniGene | Accession |
| Gene Name | (average) | Accession No. | No. |
| acyl-coenzyme A:cholesterol acyltransferase | 2.5 | Hs.14553 | NP_003092 |
| adrenomedullin | 2.3 | S73906 | AAC60642 |
| chitinase precursor = YKL-39 | 2.8 | Hs.154138 | NP_003991 |
| collagen type III, alpha 1 | 4.3 | Hs.119571 | NP_000081 |
| DNA sequence clone RP11-27N17 | 2.3 | AC021801 | |
| EST (ah53c02.s1) | 2.1 | Hs.31819 | NP_065095 |
| EST (ts23b10.x1) | 3.0 | Hs.118446 | |
| EST (zf50c04.s1) | 2.0 | Hs.77550/ | |
| | | AA047880 | |
| hypothetical protein | 3.2 | AL035369 | CAA23019 |
| hypothetical protein CGI-110 | 2.5 | Hs.177861 | NP_057131 |
| hypothetical protein (DKFZp564H122) | 3.6 | Hs.28783 | XP_048747 |
| lumican | 2.3 | Hs.79914 | NP_002336 |
| MAD-3 (IkB-like activity) | 2.6 | Hs.81328 | NP_065390 |
| manganese superoxide dismutase (SOD-2) | 2.1 | X65965 | |
| nicotinamide N-methyltransferase (NNMT) | 3.2 | Hs.364345 | NP_006160 |
| nuclear autoantigen (SP-100) | 28.0 | Hs.77617 | NP_003104 |
| syntaxin 4 binding protein UNC-18c | 7.0 | Hs.8813 | NP_009200 |
| syntaxin 7 | 3.8 | Hs.8906 | XP_098468. |
| transforming growth factor-beta induced gene | 3.6 | Hs.118787 | NP_000349 |
| product (BIGH3) | | | |
| translational inhibitor protein p14.5 | 2.3 | Hs.18426 | NP_005827 |

TABLE 3
Eleven genes down-regulated by B2M. Alphabetical
list of genes down regulated at least two-fold
by B2M in OA chondrocytes (two experiments).

| | | GeneBank or | Protein |
|---|---|---|---|
| | Fold | UniGene | Accession |
| Gene Name | (average) | Accession No. | No. |
| Asporin | 2.0 | Hs.10760 | NP_060150 |
| EST (yx42g01.s1) | 2.0 | Hs.19280 | NP_057525 |
| EST (nz94a11.s1) | 4.0 | AA730269 | JQ0129 |
| EST (zr99b03.r1) | 2.4 | Hs.14456 | NP_690869 |
| EST (zw78b10.r1) | 2.0 | Hs.70333 | NP_057712 |
| hypothetical protein | 2.7 | Hs.77665 | NP_055567 |
| (KIAA0102) | | | |
| intersectin short form | 2.4 | Hs.66392 | NP_003015 |
| KARP-1-binding protein | 2.3 | Hs.25132 | NP_055627 |
| 2 (KAB2) | | | |
| membrane protein | 2.0 | Hs.93832 | NP_061899 |
| peripheral myelin | 2.1 | Hs.103724 | NP_000295 |
| protein 22 (PMP22) | | | |
| putative GTP-binding | 2.8 | AJ006412 | CAA07018 |
| protein | | | |

What is claimed is:

1. An isolated biomarker comprising two or more genes selected from the group consisting of the 31 genes as set out in Tables 2 and 3.

2. The isolated biomarker of claim 1 consisting essentially of the 31 genes as set out in Tables 2 and 3.

3. An isolated biomarker comprising one or more polynucleotide sequences from the 5′ region of a gene selected from the group consisting of the 31 genes as set out in Tables 2 and 3.

4. An isolated biomarker comprising one or more polynucleotide sequences from the 3′ region of a gene selected from the group consisting of the genes as set out in Tables 2 and 3.

5. An isolated biomarker comprising one or more polynucleotide sequences from the internal coding region of a gene selected from the group consisting of the 31 genes as set out in Tables 2 and 3.

6. An isolated biomarker comprising the polypeptide sequences encoded by two or more genes selected from the group consisting of the 31 genes as set out in Tables 2 and 3.

7. The isolated biomarker of claim 6 consisting essentially of the polypeptide sequences encoded by the 31 genes, as set out in Tables 2 and 3.

8. An isolated biomarker comprising the amino terminal polypeptide sequences encoded by one or more polynucleotide sequences from the 5′ region of a gene selected from the group consisting of the 31 genes as set out in Tables 2 and 3.

9. An isolated biomarker comprising the carboxy terminal polypeptide sequences encoded by one or more polynucleotide sequences from the 3′ region of a gene selected from the group consisting of the 31 genes as set out in Tables 2 and 3.

10. An isolated biomarker comprising the internal polypeptide sequences encoded by one or more polynucleotide sequences from the internal coding region of a gene selected from the group consisting of the 31 genes as set out in Tables 2 and 3.

11. A method of identifying an inhibitor of B2M activity said method comprising the steps of:
a) contacting chondrocytes with B2M in the presence and absence of a candidate modulator; and
b) comparing the proliferation of said chondrocytes in the presence relative to the absence of said candidate modulator, wherein an increase in the proliferation of said chondrocytes in the presence relative to the absence of said candidate modulator identifies said candidate modulator as an inhibitor of B2M activity.

12. A method of identifying an inhibitor of B2M activity said method comprising the steps of:
a) contacting chondrocytes with B2M in the presence and absence of a candidate modulator, and
b) comparing the level of differential expression of a biomarker comprising one or more polynucleotide sequences of one or more genes selected from the group consisting of the 31 genes as set out in Tables 2 and 3 in the presence relative to the absence of said candidate modulator, wherein differentially decreased expression of said biomarker identifies said candidate modulator as an inhibitor of B2M activity.

13. The method of claim 12, wherein said polynucleotide sequences are from the 5′ region of a gene selected from the group consisting of the 31 genes as set out in Tables 2 and 3.

14. The method of claim 12, wherein said polynucleotide sequences are from the 3′ region of a gene selected from the group consisting of the 31 genes as set out in Tables 2 and 3.

15. The method of claim 12, wherein said polynucleotide sequences are from the internal coding region of a gene selected from the group consisting of the 31 genes as set out in Tables 2 and 3.

16. A method of identifying an inhibitor of B2M activity said method comprising the steps of:
a) contacting chondrocytes with B2M in the presence and absence of a candidate modulator, and
b) comparing the level of differential expression of a biomarker comprising one or more polypeptide sequences of one or more genes selected from the group consisting of the 31 genes as set out in Tables 2 and 3 in the presence relative to the absence of said candidate modulator, wherein differentially increased expression of said biomarker identifies said candidate modulator as an inhibitor of B2M or B2M related activity.

17. The method of claim 16, said polypeptide sequences are amino terminal polypeptide sequences encoded by one or more polynucleotide sequences from the 5′ region of a gene selected from the group consisting of the 31 genes as set out in Tables 2 and 3.

18. The method of claim 16, said polypeptide sequences are carboxy terminal polypeptide sequences encoded by one or more polynucleotide sequences from the 3′ region of a gene selected from the group consisting of the 31 genes as set out in Tables 2 and 3.

19. The method of claim 16, said polypeptide sequences are internal polypeptide sequences encoded by one or more polynucleotide sequences from the internal coding region of a gene selected from the group consisting of the 31 genes as set out in Tables 2 and 3.
